SEQUENCE LISTING (12 sequences)

SEQ ID NO: 1 - DNA, 318 bp, synthetic construct
  source 1..318: mol_type = other DNA
ttgaatgaggcttcagtactttacagaatcgttgcctgcacatcttggaaacacttgctgggattacttcttcaggttaacccaacagaaggctaaagaaggtatattgctgttgacagtgagcgaaggcagaagtatgcaaagcattagtgaagccacagatgtaatgctttgcatacttctgcctgtgcctactgcctcggacttcaaggggctactttaggagcaattatcttgtttactaaaactgaataccttgctatctctttgatacatttttacaaagctgaattaaaatggtataaattaaatcacttt

SEQ ID NO: 2 - DNA, 318 bp, synthetic construct
  source 1..318: mol_type = other DNA
ttgaatgaggcttcagtactttacagaatcgttgcctgcacatcttggaaacacttgctgggattacttcttcaggttaacccaacagaaggctaaagaaggtatattgctgttgacagtgagcgaagctaacatcctcatcaatgatagtgaagccacagatgtatcattgatgaggatgttagctctgcctactgcctcggacttcaaggggctactttaggagcaattatcttgtttactaaaactgaataccttgctatctctttgatacatttttacaaagctgaattaaaatggtataaattaaatcacttt

SEQ ID NO: 3 - DNA, 318 bp, synthetic construct
  source 1..318: mol_type = other DNA
ttgaatgaggcttcagtactttacagaatcgttgcctgcacatcttggaaacacttgctgggattacttcttcaggttaacccaacagaaggctaaagaaggtatattgctgttgacagtgagcgaccccaagaaggaaaagatgaatagtgaagccacagatgtattcatcttttccttcttgggggtgcctactgcctcggacttcaaggggctactttaggagcaattatcttgtttactaaaactgaataccttgctatctctttgatacatttttacaaagctgaattaaaatggtataaattaaatcacttt

SEQ ID NO: 4 - DNA, 318 bp, synthetic construct
  source 1..318: mol_type = other DNA
ttgaatgaggcttcagtactttacagaatcgttgcctgcacatcttggaaacacttgctgggattacttcttcaggttaacccaacagaaggctaaagaaggtatattgctgttgacagtgagcgatgcccttctcgtaaagttgtatagtgaagccacagatgtatacaactttacgagaagggcactgcctactgcctcggacttcaaggggctactttaggagcaattatcttgtttactaaaactgaataccttgctatctctttgatacatttttacaaagctgaattaaaatggtataaattaaatcacttt

SEQ ID NO: 5 - DNA, 296 bp, synthetic construct
  source 1..296: mol_type = other DNA
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtgacattttgacacccccataatatttttccagaattaacagtataaattgcatctcttgttcaagagttccctatcactctctttaatcactactcacagtaacctcaactcctgc

SEQ ID NO: 6 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 7 - DNA, 466 bp, synthetic construct
  source 1..466: mol_type = other DNA
ctagcaataaaagatctttattttcattagatctgtgtgttggttttttgtgtgtctagttaattaaggtggggcagtgggggccaacttgtccttacccagagtgcaggtgtgtggagatccctcctgccttgacattgagcagccttagagggtgggggaggctcaggggtcaggtctctgttcctgcttattggggagttcctggcctggcccttctatgtctccccaggtaccccagtttttctgggttcacccagagtgcagatgcttgaggaggtgggaagggactatttgggggtgtctggctcaggtgccatgcctcactggggctggttggcacctgcatttcctgggagtggggctgtctcagggtagctgggcacggtgttcccttgagtgggggtgtagtgggtgttcctagctgccacgcctttgccttcacctatgggatcgtggctgtcag

SEQ ID NO: 8 - DNA, 1059 bp, synthetic construct
  source 1..1059: mol_type = other DNA
atggcacactttcctgggtttgggcagtcactgctctttgggtaccccgtttatgtattcggcgactgtgttcaaggagactggtgtcctatctcaggcggtctctgttccgctcgactccaccggcatgcactcctcgcgacatgtcccgagcatcagataacctgggatccaatcgacggaagggtgataggctctgccctgcagtttctcatccctcgcctgcccagctttcccacccagcgaacctccaaaaccttgaaggtcttgacccctccaatcacacatactacccctaacatcccgccttccttcctgcaagccatgcgaaaatacagccccttcagaaacggttacatggaacctactttgggtcagcacctgcctacattgtcctttccggaccccggcctgcgaccgcagaacctgtatacactgtggggcggctctgtcgtctgtatgtacctctaccagctgtcaccgcccatcacgtggcccctgttgccccatgtaatattttgccacccaggacagctcggcgcctttctgacaaacgtcccttataaacgaatcgaaaaactcctgtataaaatttcactgaccaccggagccctgataatactgcctgaagactgcctgccaaccactctgtttcaacccgctagggctcccgtcaccctcactgcttggcagaacggactcctgcccttccattccaccctcacaactccagggttgatttggacatttaccgatggaactcctatgatttccggaccttgtcccaaggacggacagccatccttggtgctccaatcctcctcctttattttccacaagtttcaaacgaaggcataccatcctagcttcctgctgagccatggactgatacagtactcttcttttcataatctgcacctgttgtttgaggagtatactaatatcccaatcagcctcctctttaatgaaaaagaggcggacgacaatgatcatgagccgcagatctcacctggcgggctggagccccttagcgaaaagcattttcgcgaaacagaggtc

SEQ ID NO: 9 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 10 - DNA, 407 bp, synthetic construct
  source 1..407: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcggccgg

SEQ ID NO: 11 - DNA, 411 bp, synthetic construct
  source 1..411: mol_type = other DNA
atgaccaacatgagctgcgcctacgagctgatcaagagcctgcccgccaagctggaacagctggcccaggaaacccaggccaccatccagaccctgatgatcgccgaccccaacgtgaacaaggacctgcgggccttctgcgagttcctgaccgtgcagcaccagcgggcctacagagccaccaacagcctgctgattaagcccagagtggccgctgccctgagaggcgaggaactggatctgggagaggccgatgtggccgctagagtgcggcagctgaaacagcagctggccgagctggaaatggaaatcaagcccggccatcagcaggtggcccaggtgtccggaagaagaaaagccgccgctgccgctcctgtggctcagctgggaagagtgggagtcgtgaacgag

SEQ ID NO: 12 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

(19) United States
(12) Patent Application Publication
(10) Pub. No.: US 2025/0009802 A1
(43) Pub. Date: Jan. 9, 2025

(54) ANTISENSE TRANSFER VECTORS AND METHODS OF USE THEREOF

(71) Applicants: Ludwig Institute for Cancer Research Ltd, Zurich (CH); Centre Hospitalier Universitaire Vaudois, Lausanne (CH); University of Lausanne, Lausanne (CH)

(72) Inventors: Melita Irving, Epalinges (CH); George Coukos, Epalinges (CH); Patrick Reichenbach, La Sarraz (CH); Greta Giordano Attianese, Epalinges (CH)

(21) Appl. No.: 18/712,371

(22) PCT Filed: Dec. 15, 2022
(86) PCT No.: PCT/US22/81673

§ 371 Date: May 22, 2024

Related U.S. Application Data
(60) Provisional application No. 63/290,528, filed on Dec. 16, 2021.

(51) Int. Cl. A61K 35/17; A61K 39/00; A61P 35/00; C12N 15/113; C12N 15/86

(52) U.S. Cl. CPC A61K 35/17; A61K 39/4611; A61K 39/4631; A61K 39/464412; A61K 39/464495; A61P 35/00; C12N 15/1137; C12N 15/86; C12N 2310/141; C12N 2310/531; C12N 2740/10043; C12N 2740/15043; C12N 2740/16043; C12N 2830/205; C12N 2830/50; C12Y 207/11001

(57) ABSTRACT

The present disclosure describes a next-generation antisense transfer vector along with methods for a high titer lentivirus production, allowing efficient transduction of T cells with a constitutively expressed tumor-targeting receptor along with the activation-induced expression of various gene cargos. The disclosed antisense transfer vector and the methods can reduce virus production costs as well as enhance the efficacy and safety of next-generation CAR- or TCR-T cells reaching the clinic.

### CROSS-REFERENCE TO RELATED APPLICATIONS

This application claims priority under 35 U.S.C. § 119(e) to U.S. Provisional Patent Application No. 63/290,528, filed Dec. 16, 2021. The foregoing application is incorporated by reference herein in its entirety.

### FIELD OF THE INVENTION

The present invention relates generally to an antisense transfer vector and methods of use thereof and relates specifically to an antisense viral transfer vector and methods of use thereof, allowing efficient transduction of T cells with constitutive expression of a tumor-targeting receptor and specific expression of a gene cargo upon T-cell activation.

### BACKGROUND OF THE INVENTION

Important technological advances in recent years in the field of cellular engineering have enabled increasing clinical translation of gene-modified cells for the treatment of cancer and other diseases. Transient or stable alterations can be made to host cells, such as hematopoietic stem cells, or immune cells including T cells, B cells, natural killer cells, and macrophages, to modify their properties for a desired therapeutic outcome upon re-infusion into a patient. Disruption of cellular processes can be attained by silencing, correcting, or overexpressing targets within the genome, or by RNA interference of transcribed genes such as by short hairpin (sh)RNA or microRNA (miR; non-coding RNAs). If only temporary changes in gene expression are desired, such as for evaluating the safety of a new cellular product, messenger (m)RNA electroporation can be used, and advances in non-viral episomal vector design show promise in enabling longer-term modifications to gene expression. Whereas, for permanent modifications, a variety of tools have been developed for genome-editing including zinc finger nucleases, transcription activator like (TAL) effector nucleases, clustered regularly interspaced short palindromic repeats (CRISPR)/Cas9, and viral vectors such as adenovirus, adeno-associated virus (AAV), and retroviruses.

Both lentivirus and gamma-retrovirus are subtypes of retroviruses comprising an RNA genome that is converted to DNA in infected host cells by the virally encoded enzyme reverse transcriptase, and they allow efficient non-site-directed integration of genes of interest into the genome. Lentiviral and gamma-retroviral vector based gene-engineering strategies have been widely and safely used in the clinic for both CAR- and TCR-T-cell therapy of cancer. In particular, CAR-T cells targeting the B-cell lineage antigen CD19 have conferred unprecedent clinical responses against certain hematological malignancies such as acute lymphoblastic leukemia. In addition, TCR-engineered T cells targeting the HLA-A2 restricted cancer testis epitope NY-ESO-1₁₅₇₋₁₆₅ (A2/NY) have shown promise for the treatment of melanoma, myeloma and synovial cell sarcoma. The continued importance of lentiviral vectors as a tool for T-cell engineering purposes for clinical application is underscored by recent advances in improving CAR-T cell manufacturing protocols.

CARs are synthetic receptors that can be used in place of a TCR-CD3 complex to link tumor antigen binding and cellular activation upon target engagement in a non-major histocompatibility complex (MIC) restricted manner. While first generation (1G) CARs comprise the endodomain of CD3-zeta for signal 1 of T-cell activation, 2G and 3G CARs further include one or more costimulatory endodomains, respectively. CAR therapy has been a powerful strategy for fighting some advanced hematological malignancies, but a significant proportion of patients either do not benefit or they relapse. Moreover, epithelial-derived solid tumors remain poorly responsive to CAR therapy, and the efficacy of TCR-engineered T cells, as well as of tumor-infiltrating lymphocyte (TIL) transfer, have proven beneficial against relatively few cancer types and a modest proportion of patients. It is widely held, however, that the development of personalized combinatorial or/and co-engineering strategies to overcome barriers in the tumor microenvironment (TME) and harness endogenous immunity can further improve responses to these different T cell-based therapies. Co-engineered CAR-T cells are referred to as 4G CARs, armoured CARs or next-generation CARs. And the term TRUCK (‘T cells redirected for universal cytokine mediated killing’) has been coined to define T cells specifically engineered to enforce expression of cytokines/interleukins (ILs). Examples of cytokines evaluated in the context of CAR- and TCR-T cells, and in some instances TTLs, include IL-12, IL-15 and IL-18.

While in early studies, the co-expression of genes in T cells was achieved by dual transduction (Chmielewski, M., et al. Cancer Res 71, 5697-5706 (2011); Yeku, 0.0. & Brentjens, R. J. Biochem Soc Trans 44, 412-418 (2016)), the high cost of GMP-grade virus production and elevated risk for insertional mutagenesis have driven the development of ‘all-in-one’ multi-gene encoding vectors. If both the receptor (CAR or TCR) and the gene cargo are constitutively expressed, they can be separated on the transfer vector by an internal ribosome entry site (IRES). Alternatively, for equimolar expression of both genes, a picornavirus 2A peptide sequence (P2A) can be used. For both approaches, RNA is generated from a single promoter, and coexpression is reliant upon functioning of the interspersed element. Disadvantages of IRES are its relatively large size (about 500 bp), cell-type dependency, and reduced expression of the downstream gene. Drawbacks of P2A are the risk of incomplete cleavage and potential immunogenicity of the gene product.

Therefore, there is a strong need for a novel strategy for co-expression of genes, such as a CAR or TCR and a gene cargo in T cells.

### SUMMARY OF THE INVENTION

This disclosure addresses the need mentioned above in a number of aspects. In one aspect, this disclosure provides a polynucleotide comprising: (i) a first gene cassette comprising at least a first polynucleotide sequence operably linked to a constitutive promoter or an inducible promoter; and (ii) a second gene cassette comprising at least a second polynucleotide sequence operably linked to a second constitutive promoter or a second inducible promoter, wherein both the first gene cassette and the second gene cassette are in antisense orientation and in the same strand of the polynucleotide.

In some embodiments, the polynucleotide comprises: (i) a first gene cassette comprising at least a first polynucleotide sequence encoding a CAR (e.g., second or third generation CAR, split, remote control, and switchable CAR, a co-stimulatory CAR), a TCR or a cellular elimination tag (CET) (e.g., truncated EGFR, truncated HER2) operably linked to a constitutive promoter or an inducible promoter; and (ii) a second gene cassette comprising at least a second polynucleotide sequence encoding a gene cargo operably linked to a second constitutive promoter or a second inducible promoter, wherein both the first gene cassette and the second gene cassette are in antisense orientation and in the same strand of the polynucleotide. In some embodiments, the second inducible promoter can be induced by binding of the CAR or TCR (e.g., introduced or endogenous TCR in a TIL or a TCR that is knocked in by gene editing, e.g., CRISPR/Cas9, sleeping beauty) to a target antigen thereof.

In some embodiments, the first polynucleotide sequence is operably linked to the constitutive promoter. In some embodiments, the second polynucleotide sequence is operably linked to the second inducible promoter.

In some embodiments, the first gene cassette is located in 5′ of the second gene cassette. In some embodiments, the polynucleotide further comprises a polyadenylation (PA) signal located between the first gene cassette and the second gene cassette, whereby independent RNAs are transcribed and separately translated. In some embodiments, the first gene cassette and the second gene cassette are arranged between a 5′LTR and a 3′ LTR. In some embodiments, the 3′ LTR is a self-inactivating (SIN) LTR.

In some embodiments, the first gene cassette or the second gene cassette comprises two or more polynucleotide sequences. In some embodiments, the two or more polynucleotide sequences are separated by a T2A or P2A element.

In some embodiments, the first gene cassette further comprises a third polynucleotide sequence that is separated from the first polynucleotide sequence by, e.g., a T2A or P2A element. In some embodiments, the second gene cassette further comprises a fourth polynucleotide sequence that is separated from the second polynucleotide sequence by, e.g., a T2A or P2A element.

In some embodiments, the constitutive promoter comprises any one of a phosphoglycerate kinase-1 (PGK) promoter (e.g., human PGK (hPGK) promoter), a cytomegalovirus (CMV) immediate-early gene promoter, an elongation factor 1 alpha (EF1α) promoter, a ubiquitin-C (UBQ-C) promoter, a cytomegalovirus (CAG) enhancer/chicken beta-actin promoter, a polyoma enhancer/herpes simplex thymidine kinase (MC1) promoter, a beta-actin (β-ACT) promoter, a simian virus 40 (SV40) promoter, a dl587rev primer-binding site substituted (MND) promoter, and a combination thereof.

In some embodiments, the inducible promoter comprises an NFAT promoter (e.g., NFATc1, NFATc3, NFATc2).

In some embodiments, the CAR or TCR binds to an antigen (e.g., a tumor antigen) selected from: prostate-specific membrane antigen (PSMA), Carcinoembryonic Antigen (CEA), CD19, CD20, CD22, ROR1, mesothelin, CD333/IL3Ra, c-Met, Glycolipid F77, EGFRvIII, GD-2, NY-ESO-1 TCR, ERBB2, BIRC5, CEACAM5, WDR46, BAGE, CSAG2, DCT, MAGED4, GAGE1, GAGE2, GAGE3, GAGE4, GAGE5, GAGE6, GAGE7, GAGE 8, IL13RA2, MAGEA1, MAGEA2, MAGE A3, MAGEA4, MAGEA6, MAGEA9, MAGE A 10, MAGEA12, MAGEB1, MAGEB2, MAGEC2, TP53, TYR, TYRP1, SAGEl, SYCP1, SSX2, SSX4, KRAS, PRAME, NRAS, ACTN4, CTNNB1, CASP8, CDC27, CDK4, EEF2, FN1, HSPA1B, LPGAT1, MEl, HHAT, TRAPPC1, MUM3, MYOIB, PAPOLG, OS9, PTPRK, TPI1, ADFP, AFP, AIM2, ANXA2, ART4, CLCA2, CPSF1, PPIB, EPHA2, EPHA3, FGF5, CA9, TERT, MGAT5, CEL, F4.2, CAN, ETV6, BIRC7, CSF1, OGT, MUC1, MUC2, MUM1, CTAG1A, CTAG2, CTAG, MRPL28, FOLH1, RAGE, SFMBT1, KAAG1, SART1, TSPYL1, SART3, SOX10, TRG, WTI, TACSTD1, SILV, SCGB2A2, MC1R, MLANA, GPR143, OCA2, KLK3, SUPT7L, ARTC1, BRAF, CASP5, CDKN2A, UBXD5, EFTUD2, GPNMB, NFYC, PRDX5, ZUBR1, SIRT2, SNRPD1, HERV-K-MEL, CXorffi1, CCDC1 10, VENTXP1, SPA17, KLK4, ANKRD30A, RAB38, CCND1, CYP1B1, MDM2, MMP2, ZNF395, RNF43, SCRN1, STEAPI, 707-AP, TGFBR2, PXDNL, AKAP13, PRTN3, PSCA, RHAMM, ACPP, ACRBP, LCK, RCVRN, RPS2, RPLIOA, SLC45A3, BCL2L1, DKK1, ENAH, CSPG4, RGS5, BCR, BCR-ABL, ABL-BCR, DEK, DEK-CAN, ETV6-AML1, LDLR-FUT, NPM1-ALK1, PML-RARA, SYT-SSX1, SYT-SSX2, FLT3, ABL1, AML1, LDLR, FUT1, NPM1, ALK, PML1, RARA, SYT, SSX1, MSLN, UBE2V1, HNRPL, WHSC2, EIF4EBP1, WNK2, OAS3, BCL-2, MCL1, CTSH, ABCC3, BST2, MFGE8, TPBG, FMOD, XAGE1, RPSA, COTL1, CALR3, PA2G4, EZH2, FMNL1, HPSE, APC, UBE2A, BCAP31, TOP2A, TOP2B, ITGB8, RPA1, ABI2, CCNI, CDC2, SEPT2, STAT1, LRP1, ADAM17, JUP, DDR1, ITPR2, HMOX1, TPM4, BAAT, DNAJC8, TAPBP, LGALS3BP, PAGE4, PAK2, CDKN1A, PTHLH, SOX2, SOX11, TRPM8, TYMS, ATIC, PGK1, SOX4, TOR3A, TRGC2, BTBD2, SLBP, EGFR, IER3, TTK, LY6K, IGF2BP3, GPC3, SLC35A4, HSMD, H3F3A, ALDH1A1, MFI2, MMP14, SDCBP, PARP12, MET, CCNB1, PAX3-FKHR, PAX3, FOXO1, XBP1, SY D1, ETV5, HSPA1A, HMHA1, TRIM68, and a fragment thereof.

In some embodiments, the gene cargo is selected from IL-2, IL2v, IL-12, IL-15, IL-18, IL21, IFNγ, IL7, IL23, IL33, GM-CSF, Flt3-L, 41BB-L, CD40-L, TGFb, VEGF, IL10, PD1, TGFβR, a dominant negative receptor, a signal switch receptor, CCL5, CXCL9, CXCL10, XCL1, and a combination thereof.

In some embodiments, the first gene cassette comprises one or more genes of a CAR, a co-stimulatory CAR, a TCR, a cellular elimination tag, and a decoy that are regulated by the constitutive promoter. In some embodiments, the second gene cassette comprises one or more genes of a cytokine, a Flt3L, a LIGHT, a chemokine, a co-stimulatory ligand, a decoy, a dominant negative receptor, a signal switch receptor, and a gene knockdown that are regulated by the second inducible promoter.

In some embodiments, the first gene cassette comprises one or more genes of a cytokine, a chemokine, a co-stimulatory ligand, a decoy, a Trap, a dominant negative receptor, a signal switch receptor, and a gene knockdown that are regulated by the inducible promoter. In some embodiments, the second gene cassette comprises one or more genes of a cytokine, a chemokine, a co-stimulatory ligand, a decoy, a dominant negative receptor, a signal switch receptor, and a second gene knockdown to complement a gene in the first gene cassette, wherein the one or more genes are regulated by the second inducible promoter.

In some embodiments, the gene cargo comprises an shRNA, miRNA or a sequence enabling down-regulation of a target gene. In some embodiments, the target gene comprises HPK1 or Cblb.

In another aspect, this disclosure also provides a vector comprising a polynucleotide as described above. In some embodiments, the vector is a retroviral vector or a lentiviral vector. In some embodiments, the lentiviral vector is selected from human immunodeficiency virus 1 (HIV-1), human immunodeficiency virus 2 (HIV-2), visna-maedi virus (VMV), caprine arthritis-encephalitis virus (CAEV), equine infectious anemia virus (EIAV), and feline immunodeficiency virus (FIV).

Also provided in this disclosure is a viral particle or virus-like particle (VLP) comprising a polynucleotide as described herein.

In another aspect, this disclosure additionally provides a cell comprising a polynucleotide or a vector, as described above. In some embodiments, the cell is selected from a cytotoxic T lymphocyte (CTL), a natural killer (NK) cell, a natural killer T (NKT) cell, a tumor-infiltrating lymphocyte (TIL), a CD4T cell, a B cell, a macrophage, and a dendritic cell (DC). In some embodiments, the cell is autologous or allogeneic.

In another aspect, this disclosure further provides a pharmaceutical composition comprising a polynucleotide, a vector, a viral particle or virus-like particle, or a cell, as described herein.

Also provided in this disclosure is a kit comprising a polynucleotide, a vector, a viral particle or virus-like particle, a cell, or a pharmaceutical composition, as described herein.

In another aspect, this disclosure also provides a method for preparing an immune effector cell expressing a CAR or TCR. The method comprises introducing into an immune effector cell a polynucleotide or a vector, as described herein.

In some embodiments, the second polynucleotide is contained in an envelope vector. In some embodiments, the envelope vector comprises an env gene selected from VSV-G env, LCMV env, LCMV-GP(WE-HPI) env, MoMLV env, Gibbon Ape Leukemia Virus (GaLV) env; or an env gene selected from a member of the Pbabdoviridae, an Alphavirus env gene, a Paramyxovirus env gene, a Flavivirus env gene, a Retrovirus env gene, an Arenavirus env gene, a Parainfluenza virus env gene, a Thogoto virus env gene, a Baculovirus env gene, and a vesicular stomatitis virus G-protein (VSV-G) envelope vector.

In some embodiments, the immune effector cell is selected from a cytotoxic T lymphocyte (CTL), a natural killer (NK) cell, a natural killer T (NKT) cell, a tumor-infiltrating lymphocyte (TIL), a CD4T cell, a B cell, a macrophage, and a dendritic cell (DC).

In yet another aspect, this disclosure further provides a method of treating cancer in a subject. The method comprises administering to the subject a therapeutically effective amount of a polynucleotide, a vector, a viral particle or virus-like particle, a cell, a pharmaceutical composition, as described above, or a cell prepared by a method described herein.

In some embodiments, the cancer is selected from Wilms' tumor, Ewing sarcoma, neuroendocrine tumor, glioblastoma, neuroblastoma, melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

In some embodiments, the method further comprises administering to the subject a second therapeutic agent. In some embodiments, the second therapeutic agent is an anti-cancer or anti-tumor agent. In some embodiments, the second therapeutic agent is administered to the subject before, after, or concurrently with the vector, the viral particle or virus-like particle, the cell, or the pharmaceutical composition.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combinations of features are not found together in the same sentence, or paragraph, or section of this document.

Other features and advantages of the invention will become apparent from the following detailed description. It should be understood, however, that the detailed description and the specific is examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1a, 1b, and 1c show that dual antisense lentiviral transfer vector allows efficient constitutive expression of a transgene and controlled co-expression of an activation-inducible transgene. For all dual transfer constructs, eGFP (Gene A) expression is constitutively driven by the PGK promoter and mCherry (Gene B) by 6xNFAT. FIG. 1a (Left) shows a schematic of dual sense orientation lentiviral transfer vector post-integration in non-stimulated (top) and stimulated (middle) transduced cells. FIG. 1a (Right) shows a representative flow cytometric analysis of transfected Jurkat cells, pre- and post-stimulation. FIG. 1a (Left, bottom panel) is a schematic illustrating that the inclusion of a polyadenylation (PA) site between the two genes will abrogate virus production in the packaging cells. FIG. 1b (Left) shows a schematic of bi-directional transfer vector post-integration in non-stimulated (top) and stimulated (bottom) transduced cells. FIG. 1b (Right) shows a representative flow cytometric analysis of transduced Jurkat cells, pre- and post-stimulation. FIG. 1c (Left) shows a schematic of antisense orientation lentiviral transfer vector post-integration in non-stimulated (top) and stimulated (bottom) transduced cells. FIG. 1c (Right) shows a representative flow cytometric analysis of transduced Jurkat cells, pre- and post-stimulation. The flow cytometry plots are representative of 5 independent experiments.

FIGS. 2a, 2b, 2c, 2d, and 2e show that antisense transfer vector yields a lower lentivirus vector titer than sense vector. For all dual constructs, eGFP (Gene A) expression is constitutively driven by the PGK promoter and mCherry (Gene B) by 6xNFAT. FIG. 2a shows representative microscopy images (10× magnification) of HEK293T cells transfected with dual sense (left) versus antisense lentiviral vectors (right) for lentivirus vector production. FIG. 2b shows viral titers (Transducing Units (TU) per ml). FIG. 2c shows transduction of Jurkat cells with decreasing volumes of lentivirus vector supernatant to evaluate % eGFP expression (on day 5) by flow cytometry. Bar graphs represent the mean+/−standard error mean (S.E.M.) of technical duplicates for three independent experiments. Representative histograms of transduction with 100 μl virus supernatant are shown for dual sense (left) and antisense (right) approaches. FIG. 2d shows a schematic of dual sense (top) versus antisense (bottom) orientation lentiviral transfer vectors encoding both eGFP and mCherry. FIG. 2e is an illustration of potential Dicer-associated mechanisms in response to dsRNA, which may be limiting to lentivirus vector production in HEK293T cells.

FIGS. 3a, 3b, 3c, 3d, 3e, 3f, and 3g show rescue of low dual antisense vector lentiviral titers in the presence of NovB2 and Tax proteins. For dual constructs, eGFP (Gene A) expression is constitutively driven by the PGK promoter and mCherry (Gene B) by 6xNFAT. FIG. 3a shows a schematic of dual sense versus antisense orientation lentiviral transfer vectors encoding both eGFP and mCherry. Antisense transfer lentivirus vector was produced in the presence or absence of NovB2 (encoded on the envelope plasmid). FIG. 3b shows viral titers (Transducing Units (TU) per ml). FIG. 3c (Left) shows transduction of Jurkat cells with decreasing volumes of lentivirus vector supernatant to evaluate % eGFP expression (on day 5) by flow cytometry. Bar graphs represent the mean+/−S.E.M. of three independent experiments. FIG. 3c (Right) shows representative histograms for Jurkat cells transduced with 100 μl of lentivirus vector supernatant produced in the absence or presence of NovB2 are shown. FIG. 3d (Left) shows a schematic of dual antisense vector encoding eGFP and comprising a chimeric LTR (AU3, R, and U5) for which the Rous Sarcoma Virus (RSV) promoter and enhancer at the 5′ LTR has been substituted by the complete CMV promoter and enhancer. FIG. 3d (Right) shows schematics representing antisense lentivirus vector production in the presence or not of Tax protein (via vector co-transfection), or of NovB2 (encoded on the envelope plasmid), or of both Tax and NovB2. FIG. 3e shows transduction of Jurkat cells with decreasing volumes of lentivirus vector supernatant to evaluate % eGFP expression (on day 5) by flow cytometric analysis. The bar graph shows the mean+/−S.E.M. of three independent experiments. FIG. 3f shows viral titers (Transducing Units (TU) per ml). FIG. 3g shows representative histograms of Jurkat cells transduced with 30 μl of lentiviral supernatant are shown.

FIGS. 4a, 4b, 4c, 4d, 4e, 4f, 4g, 4h, and 4i show in vitro testing that reveals higher activation-induced expression levels of gene cargo by 4G CAR-T cells engineered with antisense versus sense lentiviral vectors. FIG. 4a shows a schematic of lentiviral vectors encoding an anti-PSMA or anti-CD19 2G CAR (gene A) under the PKG promoter and luciferase or mCherry as gene cargo (gene B) under 6xNFAT, in both sense and antisense configurations. The 2G CARs comprise a tumor-targeted scFv, the linker region of CD8a, the transmembrane region™ and endodomain (ED) of CD28, and the ED of CD3z. FIG. 4b shows transduction efficiency of primary human CD4⁺ and CD8⁺ T cells with the two different CARs and luciferase constructs as measured by cell-surface CAR staining on day 9. Shown are mean values+/−S.E.M. for T cells from three independent, healthy donors. FIG. 4c shows PSMA⁺ PC3-PIP (upper panel) or PC3-CD19⁺ engineered tumor cell lines (bottom panel) killing assay by the CAR-T cells and untransduced (UTD)-T cells as measured by the IncuCyte instrument (decrease in total green area/μm² corresponds to target cell death) over time. Shown are mean values+/−S.E.M. Symbols indicate individual donors (n=3). Statistical significance was assessed using Two-Way ANOVA and Post-hoc Tukey test versus UTD. FIG. 4d shows evaluation of luciferase expression levels (luminescence (counts)) by activated anti-PSMA- (top panel) and anti-CD19-CAR T cells (bottom panel), measured by HIDEX. Values for the assay are the mean±S.E.M. for n=3 human T cell donors. Statistical significance was assessed using One-Way ANOVA vs. tumor cells alone. FIG. 4e shows transduction efficiency of primary human CD4⁺ and CD8⁺ T cells. FIG. 4e (Left) shows percentage of CAR⁺ positive cells, and FIG. 4e (Right) shows MFI of positive cells by direct surface cell staining on day 9. FIG. 4f shows the results of PSMA⁺ PC3-PIP (left panel) killing assay by CAR- and UTD-T cells as measured by the IncuCyte instrument (total green area/μm²) overtime. Shown are mean values+/−S.E.M. Symbols indicate individual donors (n=3). Statistical significance was assessed using Two-Way ANOVA and Post-hoc Tukey test versus UTD. FIG. 4f (Right panel) shows evaluation of mCherry expression (total red area/μm²) by activated anti-PSMA tumor-cell reactive CAR-T cells. Values for the IncuCyte assay are the mean S.E.M. for n=3 human T cell donors. Statistical significance was assessed by Two-Way ANOVA and Post-hoc Tukey test. FIG. 4g shows a flow cytometric analysis to evaluate % mCherry (left) and mCherry MFI (right) background expression levels in non-activated CAR-T cells. FIG. 4h shows a flow cytometric analysis to evaluate % mCherry (left) and mCherry MFI (right) expression by activated CAR-T cells upon 24 h co-culture with PSMA⁺ PC3-PIP tumor cells. FIG. 4i shows a flow cytometric analysis to evaluate of % mCherry (left) and mCherry MFI (right) by CAR-T cells after 24 h PMA-Ionomycin stimulation. The bar graphs (FIGS. 4g-i) show the mean values+/−S.E.M. Symbols indicate individual healthy T cell donors (n=3). Statistical significance was assessed by One-Way ANOVA. (a=antisense; s=sense; mC=mCherry) FIGS. 5a, 5b, 5c, 5d, and 5e show in vivo testing that reveals higher activation-induced expression levels of gene cargo by 4G CAR-T cells engineered with antisense versus sense is lentiviral vectors. FIG. 5a shows a schematic of sense and antisense lentiviral vectors encoding anti-PSMA and anti-CD19 CARs under the PGK promoter and luciferase under 6xNFAT. FIG. 5b shows a schematic of the in vivo study. FIG. 5c shows caliper tumor volume measurements over days. Values are the mean+/−S.E.M. for n=6 mice per group. Statistical significance was determined by Two-way ANOVA. FIG. 5d is a bar graph showing the mean value of luciferase flux as measured by bioluminescence imaging upon luciferin injection for all the experimental groups. Data are represented as mean+/−S.E.M. and for n=6 mice per group. Statistical significance was assessed using Two-Way ANOVA and Post-hoc Tukey test, ns=non-significant, **=p<0.01, and ****=p<0.0001). FIG. 5e shows representative images of luciferase activity of the transferred, tumor-infiltrating 4G CAR-T cells over days upon luciferin injection of mice.

FIGS. 6a, 6b, 6c, 6d, 6e, 5f, 6g, 6h, 6i, 6j, and 6k show that optimized lentivirus vector production protocol yields high titers in the context of transfer vectors encoding microRNA (miR)-based short hairpin (sh)RNA. FIG. 6a shows a schematic of sense lentiviral transfer vector encoding a chimeric CMV promoter and enhancer at the 5′ LTR to allow enhanced replication in the presence of TNFα and eGFP. FIG. 6b shows transduction of Jurkat cells with decreasing volumes of lentivirus vector supernatant produced in the presence or not of TNFα and NovB2 and flow cytometric evaluation (on day 5) of % eGFP expression. The bar graph represents the mean of five independent experiments. FIG. 6c shows viral titers (Transducing Units (TU) per ml). FIG. 6d shows representative histograms of eGFP expression by Jurkat cells transduced with 100 μl of lentivirus vector supernatant. FIG. 6e shows a schematic of sense lentiviral transfer vector encoding miR-based shRNA targeting HPK1 (shRNA A and shRNA B) or scramble control (shRNA CTRL) under the U6 promoter, as well as truncated nerve growth factor receptor (tNGFR) and a TCR, both under the PGK promoter and separated by T2A sequences. FIG. 6f shows a Western blot analysis to evaluate HPK1 downregulation in Jurkat cells (technical replicates shown), together with β-actin control. FIG. 6g shows transduction efficiency of primary human CD4⁺ and CD8⁺ T cells with lentivirus vector supernatant produced in the presence of TNFα and NovB2. Five days post-transduction, the T cells were stained with HLA-A2/NY-ESO-1₁₅₇₋₁₆₅ tetramer and analyzed by flow cytometry. The bar graph represents the mean+/−S.E.M. of n=3 human T cell donors.

FIG. 6h shows a Western blot analysis to evaluate HPK1 down-regulation, together with β-actin control blot for n=3 human donors (HD). FIG. 6i shows a tumor cell killing assay for Nuclei red A2⁺/NY⁺ targets Me275 and A375, and Nuclei red and A2⁺/NY⁻ cell line Na8, by TCR-T cells with miR-based shRNA knockdown of HPK1, TCR-T cells comprising a scrambled miR-based shRNA (CTRL), and UTD-T cells, as measured by the IncuCyte instrument as a loss in the area over time. Shown are mean values for n=3 independent T cell donors+/−S.E.M. Statistical significance was assessed using Two-Way ANOVA and Post-hoc Tukey test versus the UTD group. FIG. 6j shows IFNγ release as measured by ELISA upon 24 h co-culture of TCR-T cells with miR-based shRNA knockdown of HPK1 T cells, CTRL- or UTD-T cells with A2⁺/NY⁺ targets Me275, A375 and Saos-2, and A2⁺/NY⁻ cell line Na8. Bar graphs represent the mean+/−S.E.M. for n=3 human T cell donors. FIG. 6k shows a bar graph representing the percent of CTV-negative cells (cells that have undergone proliferation) upon tumor stimulation (n=3). Shown are mean values for n=3 healthy T cell donors+/−S.E.M. Statistical significance was assessed using One-Way ANOVA.

FIGS. 7a, 7b, 7c, and 7d show that optimized clinical-grade protocol for high-titer lentivirus vector production can be used in the context of antisense vectors encoding miR-based shRNA. FIG. 7a (Left) shows a schematic of antisense lentiviral transfer vector encoding eGFP under PGK and a mCherry under 6xNFAT. FIG. 7a (Middle) shows transduction of Jurkat cells with titrated lentivirus vector supernatant produced in the presence or not of TNFα in combination with NovB2; flow cytometric evaluation of % eGFP expression on day 5. The bar graphs represent the mean+/−S.E.M of three independent experiments. FIG. 7c (Right) shows viral titers (Transducing Units (TU) per ml). FIG. 7b (Left) shows a schematic of dual antisense lentiviral transfer vector encoding eGFP under PGK and a miR-based shRNA under 6xNFAT. FIG. 7b (Middle) shows transduction of Jurkat cells with titrated lentivirus vector supernatant produced in the presence or not of TNFα or Tax in combination with NovB2; flow cytometric evaluation of % eGFP expression on day 5. The bar graph represents the mean+/−S.E.M of five independent experiments. FIG. 7b (Right) shows viral titers (Transducing Units (TU) per ml). FIG. 7c (Left) shows a schematic of antisense lentiviral transfer vector encoding an anti-PSMA-CAR under PGK and miRNA under 6xNFAT. FIG. 7c (Middle) shows transduction efficiency of primary human CD4⁺ and CD8⁺ T cells with lentivirus vector supernatant produced in the presence of TNFα and NovB2. T cells were stained with fluorescenated anti-Fab Ab to evaluate cell-surface CAR expression on day 5 post-infection. The bar graph represents the mean+/−S.E.M. of n=4 human T cell donors. FIG. 7c (Right) shows a Western blot analysis showing specific downregulation of HPK1 upon 6 hours stimulation with plate-coated anti-F(ab)₂, together with β-actin control blot of n=2 human T cell donors. FIG. 7d (Top left) shows a schematic of antisense lentiviral transfer vector encoding eGFP under PGK and miR-based shRNA targeting TRAC, or control (CTRL) miR-based shRNA, under the constitutive promoter SFFV. FIG. 7d (Bottom left) shows a representative dot plot of flow cytometric evaluation of % eGFP expression on day 5 and PAN anti-TCR antibody staining to evaluate TCR knockdown. FIG. 7d (Top right) shows transduction of Jurkat cells with different amounts of lentivirus vector supernatant. The bar graph represents the mean+/−S.E.M. of eGFP⁺ cells. FIG. 7d (Bottom right) shows the percentage of TCR⁺ cells for three independent experiments.

FIGS. 8a and 8b show that antisense lentiviral vectors overcome the transcriptional interference that occurs for dual gene-cassette sense vectors. FIG. 8a shows a representative flow cytometric analysis to evaluate expression levels (MFI) of eGFP (gene A) and mCherry (gene B) in activated Jurkat cells transduced with (top) single gene sense vectors in comparison to (FIG. 8b) sense (top) and antisense (bottom) dual gene cassette antisense vectors. Vector schematics are shown next to each plot. Plots are representative of three independent experiments, each performed in replicate.

FIGS. 9a and 9b show higher gene expression levels in Jurkat cells transduced with dual antisense versus sense lentiviral vectors comprising inducible gene cargo. For all dual transfer constructs, eGFP (Gene A) expression is constitutively driven by the PGK promoter and mCherry (Gene B) by 6xNFAT, as shown in the vector schematics on the top of FACS plots. FIG. 9a shows representative flow cytometry dot plots for nonstimulated and stimulated Jurkat cells transduced with dual sense (left) versus antisense (right) orientation lentiviral vectors. Each FACS dot plot set corresponds to an independent experiment (total independent experiments=5). FIG. 9b is a bar graph representing the Mean Fluorescence Intensity (MFI) for eGFP and mCherry in stimulated Jurkat cells transduced with sense (‘s’) versus antisense (‘a’) constructs.

FIGS. 10a and 10b show that antisense lentiviral transfer vector yields lower lentiviral titer than sense transfer vector, which can be partially restored by NovB2. FIG. 10a (Top left) shows a schematic of sense and antisense constructs encoding eGFP only. FIG. 10a (Top right) shows titer measurement expressed as Transducing Units (TU) per ml, for two independent experiments. FIG. 10a (Bottom left) shows transduction of Jurkat cells with decreasing volumes of lentivirus vector supernatant to evaluate % eGFP expression by flow cytometric analysis on day 5. The bar graph represents the mean of two independent experiments. FIG. 10a (Bottom right) shows representative histograms of Jurkat cells transduced with 30 μl sense and antisense lentivirus vector supernatant. FIG. 10b (Top left) shows a schematic of sense and antisense orientation lentiviral transfer vectors encoding eGFP post-integration in transduced cells. Antisense lentiviral vector was produced in the absence or presence of NovB2 (encoded on the envelope plasmid). FIG. 10b (Top right) shows titer measurement expressed as Transducing Units (TU) per ml for two independent experiments. FIG. 10b (Bottom left) shows transduction of Jurkat cells with decreasing volumes of lentivirus vector supernatant to evaluate % eGFP expression by flow cytometric analysis on day 5. The bar graph shows the mean of two independent experiments. FIG. 10b (Bottom right) shows representative histograms of Jurkat cells transduced with 30 μl anti-sense lentiviral vector supernatant produced in the absence or presence of NovB2.

FIGS. 11a, 11b, 11c, 11d, 11e, 11f, 11g, 11h, and 11i show that higher levels of inducible gene cargo are produced by TCR-T cells transduced with the dual antisense versus sense lentiviral vector. FIG. 11a shows a schematic of sense and antisense constructs encoding an HLA-A2 restricted NY-ESO₁₅₇₋₁₆₅ specific TCR (Gene A) 1 under the control of the PGK promoter and mCherry or hIL-2 (Gene B) under the 6xNFAT promoter. FIG. 11b (Top and bottom left) shows percentage of TCR expression as measured by tetramer staining of primary human CD4⁺ and CD8⁺ T cells transduced with sense and antisense lentivirus vector supernatant produced in the presence of TNFα and NovB2. FIG. 11b (Top and bottom right) shows TCR expression levels (MFI values) for primary human CD4⁺ and CD8⁺ T cells transduced with sense and antisense lentivirus vector supernatant produced in the presence of TNFα and NovB2. The bar graph shows the mean+/−S.E.M. for n=6 human donors for two independent experiments (n=3 per experiment). FIG. 11c shows a killing assay for TCR-T cells and UTD T cells against A2⁺/NY⁺ Saos-2 tumor cells labeled with nuclei green at a ratio of 2:1 as measured by the IncuCyte instrument over time. Loss of total green area/μm² is proportional to killing activity. Shown are mean values+/−S.E.M. for T cells from n=3 human donors. FIG. 11d shows IFNγ quantification by ELISA assay of TCR- and UTD-T cells co-cultured with A2⁺/NY⁺ Saos-2 tumor cells at a ratio of 2:1. Shown are mean values+/−S.E.M. for T cells from n=6 human donors for two independent experiments. FIG. 11e shows human (h)IL-2 quantification by ELISA assay of TCR- and UTD-T cells co-cultured with A2⁺/NY⁺ Saos-2 tumor cells at a ratio of 2:1. Shown are mean values+/−S.E.M. for T cells from is n=6 human donors for two independent experiments. FIG. 11f shows hIL-2 quantification by ELISA assay of TCR- and UTD-T cells cultured overnight in the presence of PMA-Ionomycin. FIG. 11g shows induced mCherry expression by TCR- and UTD-T cells against A2⁺/NY⁺ Saos-2 tumor cells at a ratio of 2:1 as measured by the IncuCyte instrument (total red area/μm²) over time. Shown are mean values+/−S.E.M. for T cells from n=6 human donors. FIG. 11h shows a flow cytometric analysis of mCherry expression for TCR- and UTD-T cells co-cultured with A2⁺/NY⁺ Saos-2 tumor cells at a ratio of 2:1. Shown are mean values+/−S.E.M. for T cells from n=3 human donors. FIG. 11h (Left) shows percentage of mCherry⁺ cells. FIG. 11h (Right) shows mCherry expression levels (MFI). FIG. 11i shows a flow cytometric analysis of mCherry expression for TCR- and UTD-T cells after overnight stimulation with PMA-Ionomycin. Shown are the mean values+/−S.E.M. for T cells from n=3 human donors. FIG. 11i(Left) shows percentage of mCherry⁺ cells. FIG. 11i (Right) shows mCherry expression levels (relative MFI). Two-way (FIGS. 11c and 11g) and one-way Anova (FIGS. 11b, 11d, 11e, 11f, 11h, and 11i) tests were used to determine statistical significance.

FIGS. 12a, 12b, 12c, 12d, 12e, 12f, 12g, 12h, 12i, and 12j show that T cells transduced with antisense lentiviral vector encoding a CAR and inducible gene cargo demonstrate specific in vitro and in vivo function and are not impacted by the use of NovB2 and Tax during virus production. FIG. 12a shows a schematic of sense and antisense lentiviral vectors encoding the anti-PSMA and anti-CD19 CARs under the PGK promoter and firefly luciferase under 6xNFAT. FIG. 12b (Left) shows transduction efficiency of CD4⁺ and CD8⁺ primary T cells as measured by cell-surface CAR expression. Bar graphs show the mean+/−S.E.M. of the percentage of CAR⁺ T cells. Data are for T cells from n=6 healthy donors and symbols on the graphs represent individual donors. FIG. 12b (Right) shows mCherry expression at 12 h post PMA-Ionomycin stimulation by equivalently transduced T cells as measured by flow cytometric analysis. With the T cells normalized to approximately 40% cell surface CAR expression, the graph indicates that all transduced T cells express mCherry upon activation by PMA-Ionomycin. Shown are mean values+/−S.E.M. Symbols indicate individual donors (n=3). FIG. 12c shows a schematic of CAR-T cell transfer study in PSMA⁺ PC3-PIP tumor-bearing mice. FIG. 12d shows caliper tumor volume measurements over days. Values are the mean+/−S.E.M. for n=5 mice per group. Statistical significance was determined by Two-way ANOVA. FIG. 12e shows representative images of luciferase activity of the transferred T cells over days upon luciferin injection in mice. FIG. 12f shows a bar graph showing the mean value of luciferase flux for all experimental groups. Data are represented as the mean+/−S.E.M. and for n=5 mice per group. Statistical significance was assessed using a Two-Way ANOVA and Post-hoc Tukey test. FIG. 12g shows a schematic of CAR-T cell transfer study in CD19⁺ Bjab tumor-bearing mice. FIG. 12h shows caliper tumor volume measurements over days. Values are the mean+/−S.E.M. for n=6 mice per group. Statistical significance was determined by Two-way ANOVA. FIG. 12i shows representative images of luciferase activity of the transferred T cells over days upon luciferin injection in mice. FIG. 12j shows a bar graph showing the mean value of luciferase flux for all the experimental groups. Data are represented as the mean+/−S.E.M. and for n=6 mice per group. Statistical significance was assessed using Two-Way ANOVA and Post-hoc Tukey test. (ns=non-significant, **=p<0.01 and ****=p<0.0001)

FIGS. 13a, 13b, 13c, 13d, 13e, 13f, 13g, and 13h show that the production of antisense lentiviral vector in the presence of NovB2 and Tax does not impact the activity levels of transduced T cells. FIG. 13a shows a schematic of antisense lentiviral vector encoding the anti-PSMA (Gene A) and anti-CD19 (Gene B) CARs under the PGK promoter along with mCherry under 6xNFAT. FIG. 13b shows transduction efficiency of CD4⁺ and CD8⁺ primary T cells using lentiviral supernatant produced in the absence or presence of both NovB2 and Tax. Bar graphs show the mean+/−S.E.M. of percentage of CAR⁺ T cells. Data shown are for T cells from n=3 human donors and symbols represent individual donors. FIG. 13c shows evaluation of mCherry expression (total red area/μm²) by activated anti-PSMA (left), and FIG. 13d shows anti-CD19 (right) CAR-T cells upon co-culture with PSMA+PC3-PIP tumor cells. Values for the IncuCyte assay are the mean±S.E.M. for T cells from n=3 human donors. Statistical significance was assessed using a Two-Way ANOVA and Post-hoc Tukey test. FIG. 13e shows a schematic of antisense lentiviral vectors encoding the anti-PSMA or anti-CD19 CARs (Gene A) and luciferase as gene cargo (Gene B). The CARs are expressed under the PGK promoter and luciferase under 6xNFAT. FIG. 13f shows induction of luciferase in anti-CD19 CAR-T cells upon 24 h co-culture with PC3-CD19⁺ tumor cells. The bar graph represents mean+/−S.E.M. of luminescence (counts) measured by HIDEX. Data are for T cells from n=3 human donors. FIG. 13g shows a schematic of CAR-T cell transfer study in PC3-CD19 tumor-bearing mice. FIG. 13h shows caliper tumor volume measurements over days. Values are the mean+/−S.E.M. for n=6 mice per group. Statistical significance was determined by Two-way ANOVA.

FIGS. 14a, 14b, and 14c show that TNFα can be used instead of Tax to augment transcription from vectors comprising a CMV promoter. FIG. 14a (Left) shows a schematic of pcDNA plasmid encoding eGFP under a CMV promoter in the sense orientation. FIG. 14a (Middle) is a bar graph representing % eGFP expressing HEK293T cells 48 hours after transfection with suboptimal levels of plasmid in the presence or not of co-transfected plasmid encoding Tax, soluble TNFα, or PMA. FIG. 14 (Right) is a bar graph showing relative mean fluorescence intensity (MFI) of eGFP under the different experimental conditions (eGFP encoding plasmid alone is set to 100%). FIG. 14b shows a schematic of sense lentiviral vector encoding eGFP and produced in the absence or presence of TNFα. FIG. 14c (Left) shows titer measurement expressed as Transducing Units (TU) per ml for three independent experiments. FIG. 14c (Middle) shows transduction of Jurkat cells with decreasing volumes of lentivirus vector supernatant to evaluate percentage eGFP expression by flow cytometric analysis on day 5. The bar graph represents the mean of three independent experiments. FIG. 14c (Right) shows representative histograms of Jurkat cells transduced with 30 μl sense and antisense lentivirus vector supernatant.

### DETAILED DESCRIPTION OF THE INVENTION

Innovative technological advances in cellular engineering are reshaping the clinical landscape. Chimeric antigen receptor (CAR-) T-cell therapy, for example, has conferred unprecedented responses in some treatment-refractory, advanced hematological cancer patients. Epithelial-derived solid tumors, however, remain an important challenge due to the presence of suppressive barriers in the microenvironment. It is now widely accepted that combinatorial, co-engineering strategies, along with rigorous safety mechanisms, are essential for clinical efficacy. Here, the present disclosure describes a next-generation antisense transfer vector along with methods for a high titer lentivirus production, allowing efficient transduction for constitutive CAR or TCR expression and specific expression of the gene cargo upon T-cell activation. The disclosed antisense transfer vector and the methods can reduce virus production costs as well as enhance the efficacy and safety of next-generation CAR- or TCR-T cells reaching the clinic.

### Antisense Transfer Vector and Use Thereof

In one aspect, this disclosure provides a polynucleotide, comprising: (i) a first gene cassette comprising at least a first polynucleotide sequence operably linked to a constitutive promoter or an inducible promoter; and (ii) a second gene cassette comprising at least a second polynucleotide sequence operably linked to a second constitutive promoter or a second inducible promoter, wherein both the first gene cassette and the second gene cassette are in antisense orientation and in the same strand of the polynucleotide.

In some embodiments, the polynucleotide comprises: (i) a first gene cassette comprising at least a first polynucleotide sequence encoding a CAR (e.g., second or third generation CAR, split, remote control, and switchable CAR, a co-stimulatory CAR), a TCR or a cellular elimination tag (CET) (e.g., truncated EGFR, truncated HER2) operably linked to a constitutive promoter or an inducible promoter; and (ii) a second gene cassette comprising at least a second polynucleotide sequence encoding a gene cargo operably linked to a second constitutive promoter or a second inducible promoter, wherein both the first gene cassette and the second gene cassette are in antisense orientation and in the same strand of the polynucleotide. In some embodiments, the second inducible promoter can be induced by binding of the CAR or TCR (e.g., introduced or endogenous TCR in a TIL or a TCR knocked in by gene editing, e.g., CRISPR/Cas9, sleeping beauty) to a target antigen thereof.

In some embodiments, the first polynucleotide sequence is operably linked to the constitutive promoter. In some embodiments, the second polynucleotide sequence is operably linked to the second inducible promoter.

The first gene cassette may be located in 5′ or 3′ of the second gene cassette. In some embodiments, the first gene cassette is located in 5′ of the second gene cassette. In some embodiments, the polynucleotide further comprises a polyadenylation (PA) signal located between the first gene cassette and the second gene cassette, whereby independent RNAs are transcribed and separately translated. In some embodiments, the first gene cassette and the second gene cassette are arranged between a 5′LTR and a 3′ LTR. In some embodiments, the 3′ LTR is a self-inactivating (SIN) LTR, e.g., a SIN lentivirus LTR.

In some embodiments, the first gene cassette or the second gene cassette comprises two or more polynucleotide sequences. In some embodiments, the two or more polynucleotide sequences are separated by a T2A or P2A element.

In some embodiments, the first gene cassette further comprises a third polynucleotide sequence that is separated from the first polynucleotide sequence by, e.g., a T2A or P2A element. In some embodiments, the second gene cassette further comprises a fourth polynucleotide sequence that is separated from the second polynucleotide sequence by, e.g., a T2A or P2A element.

Examples of combinations of genes that can be harbored respectively in the first gene cassette A and the second gene cassette are listed in Tables 1 and 2.

TABLE 1
Examples of antisense lentiviral vector engineering combinations
comprising constitutive Gene(s) A and inducible Gene(s) B.

Constitutive Gene(s) A
(single or multiple separated by T2A, P2A)

CAR
TCR
Cellular Elimination Tag (CET, tEGFR, tHER2, etc)
CET & PD1 decoy
Co-stimulatory CAR

Inducible Gene(s) B
(single or multiple separated by T2A, P2A)

Cytokines: IL2, IL2v, IL7, IL15, IL18, IL21, IL23, IL33, IFN, GM-CSF,
Flt3L, LIGHT (TNSF14)
Chemokines: CCL5, CXCL9, CXCL10, XCL1
Co-stimulatory ligands: 41BBL, CD40L
Decoys: PD1
Traps for: VEGF, IL10, TGFb
Dominant negative or signal switch receptors: TGFb-R, PD1-R, TIGIT-R,
IL4-R ectodomains fused either with non-signaling endodomains or to
co-stimulatory endodomains such as from CD28 or 4-1BB, etc.)
Gene Knockdown: HPK1, Cbl-b, etc.

TABLE 2
Examples of antisense lentiviral vector engineering combinations comprising inducible
Gene(s) A and inducible Gene(s) B. Here tumor antigen specificity is driven by
an endogenous TCR (e.g., TILs) or via Crispr/Cas9 TCR or CAR engineering.

| Inducible Gene(s) A | Inducible Gene(s) B |
|---|---|
| (single or multiple separated by T2A, P2A) | (single or multiple separated by T2A, P2A) |
| Cytokines: IL2, IL2v, IL7, IL15, IL18, IL21, | Cytokine(s), Chemokine(s), Co-stimulatory |
| IL23, IL33, IFN, GM-CSF, Flt3L, LIGHT | Ligand(s), Decoy(s), Trap(s), Dominant |
| Chemokines: CCL5, CXCL9, CXCL10, | negative or signal switch receptor(s) or Gene |
| XCL1 | Knockdowns to complement Gene(s) A |
| Co-stimulatory ligands: 41BBL, CD40L | selected for vector. |
| Decoys: PD1 | |
| Traps: VEGF, IL10, TGFb | |
| Dominant negative receptors or signal | |
| switch receptors: TGFb-R (TGFbR-IL7R | |
| edodomain), PD1-R (PD1R-IL7R | |
| endodomain or 41BB endodomain, etc.), | |
| TIGIT-R, IL4-R | |
| Gene Knockdowns: HPK1, Cbl-b, etc | |

In some embodiments, the constitutive promoter comprises any one of a phosphoglycerate kinase-1 (PGK) promoter (e.g., human PGK (hPGK) promoter), a cytomegalovirus (CMV) immediate-early gene promoter, an elongation factor 1 alpha (EF1α) promoter, a ubiquitin-C (UBQ-C) promoter, a cytomegalovirus (CAG) enhancer/chicken beta-actin promoter, a polyoma enhancer/herpes simplex thymidine kinase (MC1) promoter, a beta-actin (β-ACT) promoter, a simian virus 40 (SV40) promoter, a dl587rev primer-binding site substituted (MND) promoter, and a combination thereof.

In some embodiments, the inducible promoter comprises an NFAT promoter (e.g., NFATc1, NFATc3, NFATc2). Other examples of the inducible promoter that can be used in the present disclosure may include those described, for example, in U.S. Patent Publication No. 20200095573, the relevant portion of which is hereby incorporated by reference.

The terms “gene cassette” and “expression cassette” are used interchangeably, which refer to an element containing a foreign gene and having elements in addition to the foreign gene that allow for enhanced expression of that gene in a foreign host. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “promoter” or “regulatory sequence” refers to a nucleic acid sequence that is required for expression of a gene product operably linked to the promoter/regulatory sequence. In some instances, this sequence may be the core promoter sequence, and in other instances, this sequence may also include an enhancer sequence and other regulatory elements that are required for expression of the gene product. The promoter or regulatory sequence may, for example, be one that expresses the gene product in a tissue-specific manner. An “inducible” promoter is a nucleotide sequence that, when operably linked with a polynucleotide that encodes or specifies a gene product, causes the gene product to be produced in a cell substantially only when an inducer that corresponds to the promoter is present in the cell.

The term “enhancer,” as used herein, refers to a cis-acting regulatory sequence (e.g., 50-1,500 base pairs) that bind one or more proteins (e.g., activator proteins or transcription factors) to increase transcriptional activation of a nucleic acid sequence. Enhancers can be positioned up to 1,000,000 base pairs upstream of the gene start site or downstream of the gene start site that they regulate. An enhancer can be positioned within an intronic region or in the exonic region of an unrelated gene.

The term “operably linked” refers to a functional linkage between a regulatory sequence and a heterologous nucleic acid sequence resulting in expression of the latter. For example, a first nucleic acid sequence is operably linked with a second nucleic acid sequence when the first nucleic is acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary, to join two protein-coding regions in the same reading frame.

In some embodiments, the CAR or TCR binds to an antigen (e.g., a tumor antigen) selected from: prostate-specific membrane antigen (PSMA), Carcinoembryonic Antigen (CEA), CD19, CD20, CD22, ROR1, mesothelin, CD333/IL3Ra, c-Met, Glycolipid F77, EGFRvIII, GD-2, NY-ESO-1 TCR, ERBB2, BIRC5, CEACAM5, WDR46, BAGE, CSAG2, DCT, MAGED4, GAGE1, GAGE2, GAGE3, GAGE4, GAGE5, GAGE6, GAGE7, GAGE 8, IL13RA2, MAGEA1, MAGEA2, MAGE A3, MAGEA4, MAGEA6, MAGEA9, MAGE A 10, MAGEA12, MAGEB1, MAGEB2, MAGEC2, TP53, TYR, TYRP1, SAGEl, SYCP1, SSX2, SSX4, KRAS, PRAME, NRAS, ACTN4, CTNNB1, CASP8, CDC27, CDK4, EEF2, FN1, HSPA1B, LPGAT1, MEl, HHAT, TRAPPC1, MUM3, MYOIB, PAPOLG, OS9, PTPRK, TPI1, ADFP, AFP, AIM2, ANXA2, ART4, CLCA2, CPSF1, PPIB, EPHA2, EPHA3, FGF5, CA9, TERT, MGAT5, CEL, F4.2, CAN, ETV6, BIRC7, CSF1, OGT, MUC1, MUC2, MUM1, CTAG1A, CTAG2, CTAG, MRPL28, FOLH1, RAGE, SFMBT1, KAAG1, SART1, TSPYL1, SART3, SOX10, TRG, WTI, TACSTD1, SILV, SCGB2A2, MC1R, MLANA, GPR143, OCA2, KLK3, SUPT7L, ARTC1, BRAF, CASP5, CDKN2A, UBXD5, EFTUD2, GPNMB, NFYC, PRDX5, ZUBR1, SIRT2, SNRPD1, HERV-K-MEL, CXorffi1, CCDC1 10, VENTXP1, SPA17, KLK4, ANKRD30A, RAB38, CCND1, CYP1B1, MDM2, MMP2, ZNF395, RNF43, SCRN1, STEAPI, 707-AP, TGFBR2, PXDNL, AKAP13, PRTN3, PSCA, RHAMM, ACPP, ACRBP, LCK, RCVRN, RPS2, RPLIOA, SLC45A3, BCL2L1, DKK1, ENAH, CSPG4, RGS5, BCR, BCR-ABL, ABL-BCR, DEK, DEK-CAN, ETV6-AML1, LDLR-FUT, NPM1-ALK1, PML-RARA, SYT-SSX1, SYT-SSX2, FLT3, ABL1, AML1, LDLR, FUT1, NPM1, ALK, PML1, RARA, SYT, SSX1, MSLN, UBE2V1, HNRPL, WHSC2, EIF4EBP1, WNK2, OAS3, BCL-2, MCL1, CTSH, ABCC3, BST2, MFGE8, TPBG, FMOD, XAGE1, RPSA, COTL1, CALR3, PA2G4, EZH2, FMNL1, HPSE, APC, UBE2A, BCAP31, TOP2A, TOP2B, ITGB8, RPA1, ABI2, CCNI, CDC2, SEPT2, STAT1, LRP1, ADAM17, JUP, DDR1, ITPR2, HMOX1, TPM4, BAAT, DNAJC8, TAPBP, LGALS3BP, PAGE4, PAK2, CDKN1A, PTHLH, SOX2, SOX11, TRPM8, TYMS, ATIC, PGK1, SOX4, TOR3A, TRGC2, BTBD2, SLBP, EGFR, IER3, TTK, LY6K, IGF2BP3, GPC3, SLC35A4, HSMD, H3F3A, ALDH1A1, MFI2, MMP14, SDCBP, PARP12, MET, CCNB1, PAX3-FKHR, PAX3, FOXO1, XBP1, SY D1, ETV5, HSPA1A, HMHA1, TRIM68, and a fragment thereof.

In some embodiments, the gene cargo is selected from IL-2, IL2v, IL-12, IL-15, IL-18, IL21, IFNγ, IL7, IL23, IL33, GM-CSF, Flt3-L, 41BB-L, CD40-L, TGFb, VEGF, IL10, PD1, TGFβR, a dominant negative receptor, a signal switch receptor, CCL5, CXCL9, CXCL10, XCL1, and a combination thereof.

In some embodiments, the first gene cassette comprises one or more genes of a CAR, a co-stimulatory CAR, a TCR, a cellular elimination tag, and a decoy that are regulated by the constitutive promoter. In some embodiments, the second gene cassette comprises one or more genes of a cytokine, a Flt3L, a LIGHT, a chemokine, a co-stimulatory ligand, a decoy, a dominant negative receptor, signal switch receptor, and a gene knockdown that are regulated by the second inducible promoter. A “gene knockdown,” as used herein, refers to a sequence enabling down-regulation of a target gene.

In some embodiments, the first gene cassette comprises one or more genes of a cytokine, a chemokine, a co-stimulatory ligand, a decoy, a Trap, a dominant negative receptor, a signal switch receptor, and a gene knockdown that are regulated by the inducible promoter. In some embodiments, the second gene cassette comprises one or more genes of a cytokine, a chemokine, a co-stimulatory ligand, a decoy, a dominant negative receptor, a signal switch receptor, and a second gene knockdown to complement a polynucleotide sequence in the first gene cassette, wherein the one or more genes are regulated by the second inducible promoter.

In some embodiments, the gene cargo comprises a shRNA, miRNA or a sequence enabling down-regulation of a target gene. In some embodiments, the target gene comprises HPK1 or Cb1b.

In another aspect, this disclosure also provides a vector comprising a polynucleotide as described above. In some embodiments, the vector is a retroviral vector or a lentiviral vector. In some embodiments, the lentiviral vector is selected from human immunodeficiency virus 1 (HIV-1), human immunodeficiency virus 2 (HIV-2), visna-maedi virus (VMV), caprine arthritis-encephalitis virus (CAEV), equine infectious anemia virus (EIAV), and feline immunodeficiency virus (FIV).

The term “vector” or “expression vector” is synonymous with “expression construct” and refers to a nucleic acid molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery.

“Viral transfer vector” refers to a viral vector that has been adapted to deliver a gene cargo (e.g., transgene) as provided herein. “Viral vector” refers to all of the viral components of a viral transfer vector that delivers a transgene. Viral vectors are engineered to transduce one or more desired nucleic acids into a cell. The transgene may be a gene expression modulating transgene. In some embodiments, the transgene is one that encodes a protein provided herein, such as a therapeutic protein, a DNA-binding protein, etc. In other embodiments, the transgene is one that encodes an antisense nucleic acid, snRNA, an RNAi molecule (e.g., dsRNAs or ssRNAs), miRNA, or triplex-forming oligonucleotides (TFOs), etc. Viral vectors can be based on, without limitation, retroviruses (e.g., murine retrovirus, avian retrovirus, Moloney murine leukemia virus (MoMuLV), Harvey murine sarcoma virus (HaMuSV), murine mammary tumor virus (MuMTV), gibbon ape leukemia virus (GaLV) and Rous Sarcoma Virus (RSV)), lentiviruses, herpes viruses, adenoviruses, adeno-associated viruses, alphaviruses, etc. Other examples are provided elsewhere herein or are known in the art. The viral vectors may be based on natural variants, strains, or serotypes of viruses, such as any one of those provided herein. The viral vectors may also be based on viruses selected through molecular evolution. The viral vectors may also be engineered vectors, recombinant vectors, mutant vectors, or hybrid vectors. In some embodiments, the viral vector is a “chimeric viral vector.” In such embodiments, it means that the viral vector is made up of viral components that are derived from more than one virus or viral vector.

In some embodiments, the viral transfer vectors provided herein may be based on a retrovirus. Retrovirus is a single-stranded positive-sense RNA virus capable of infecting a wide variety of host cells. Upon infection, the retroviral genome integrates into the genome of its host cell, using its own reverse transcriptase enzyme to produce DNA from its RNA genome. The viral DNA is then replicated along with host cell DNA, which translates and transcribes the viral and host genes. A retroviral vector can be manipulated to render the virus replication-incompetent. As such, retroviral vectors are thought to be particularly useful for stable gene transfer in vivo. Examples of retroviral vectors can be found, for example, in U.S. Publication Nos. 20120009161, 20090118212, and 20090017543, the viral vectors and methods of their making being incorporated by reference herein in their entirety.

Lentiviral vectors are examples of retroviral vectors that can be used for the production of a viral transfer vector, as provided herein. Lentiviruses have the ability to infect non-dividing cells, a property that constitutes a more efficient method of a gene delivery vector (see, e.g., Durand et al., Viruses. 2011 February; 3(2): 132-159). Examples of lentiviruses include HIV (humans), simian immunodeficiency virus (SIV), feline immunodeficiency virus (FIV), equine infectious anemia virus (EIAV), and visna virus (ovine lentivirus). Unlike other retroviruses, HIV-based vectors are known to incorporate their passenger genes into non-dividing cells. Examples of lentiviral vectors can be found, for example, in U.S. Publication Nos. 20150224209, 20150203870, 20140335607, 20140248306, 20090148936, and 20080254008, the viral vectors and methods of their making being incorporated by reference herein in their entirety.

Also provided in this disclosure is a viral particle or virus-like particle (VLP) comprising a polynucleotide as described above. “Virus-like particle,” as used herein, refers to a structure resembling a virus particle but which has been demonstrated to be non-pathogenic. In general, virus-like particles lack at least part of the viral genome. Also, virus-like particles can often be produced in large quantities by heterologous expression and can be easily purified. A virus-like particle in accordance with the disclosure may contain nucleic acid distinct from their genome. A typical and preferred embodiment of a virus-like particle in accordance with the present invention is a viral capsid, such as the viral capsid of the corresponding virus, bacteriophage, or RNA-phage.

In another aspect, this disclosure additionally provides a cell comprising a polynucleotide or a vector, as described above. In some embodiments, the cell is selected from a cytotoxic T lymphocyte (CTL), a natural killer (NK) cell, a natural killer T (NKT) cell, a tumor-infiltrating lymphocyte (TIL), a CD4T cell, a B cell, a macrophage, and a dendritic cell (DC). In some embodiments, the cell is autologous or allogeneic.

In another aspect, this disclosure further provides a pharmaceutical composition comprising a polynucleotide, a vector, a viral particle or virus-like particle, or a cell, as described above.

In another aspect, the above-described polynucleotide, vector, viral particle or virus-like particle or cell can be incorporated into pharmaceutical compositions suitable for administration. The pharmaceutical compositions generally comprise substantially isolated/purified polynucleotide, vector, viral particle or virus-like particle or cell and optionally a pharmaceutically acceptable carrier in a form suitable for administration to a subject. Pharmaceutically-acceptable carriers are determined in part by the particular composition being administered, as well as by the particular method used to administer the composition. The pharmaceutical compositions are generally formulated in full compliance with all Good Manufacturing Practice (GMP) regulations of the U.S. Food and Drug Administration.

The terms “pharmaceutically acceptable,” “physiologically tolerable,” as referred to compositions, carriers, diluents, and reagents, are used interchangeably and include materials that are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable and includes excipients that are acceptable for veterinary use as well as for human pharmaceutical use.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the disclosed composition, use thereof in the compositions is contemplated. In some embodiments, a second therapeutic agent, such as an anti-cancer or anti-tumor agent, can also be incorporated into pharmaceutical compositions.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, is ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion, and by the use of surfactants.

Also provided in this disclosure is a kit comprising a polynucleotide, a vector, a viral particle or virus-like particle, a cell, or a pharmaceutical composition, as described above. The components of the kit may be provided in any form, e.g., liquid, dried or lyophilized form, preferably substantially pure and/or sterile. When the components of the kit are provided in a liquid solution, the liquid solution preferably is an aqueous solution. When the agents are provided as a dried form, reconstitution generally is by the addition of a suitable solvent and acidulant. The acidulant and solvent, e.g., an aprotic solvent, sterile water, or a buffer, can optionally be provided in the kit. In some embodiments, the kit may further include informational materials. The informational material of the kits is not limited in its form. For example, the informational material can include information about the production of the composition, concentration, date of expiration, batch or production site information, and so forth.

The containers can include a unit dosage of the pharmaceutical composition. In addition to the composition, the kit can include other ingredients, such as a solvent or buffer, an adjuvant, a stabilizer, or a preservative. The kit optionally includes a device suitable for administration of the composition, e.g., a syringe or other suitable delivery device. The device can be provided pre-loaded with one or both of the agents or can be empty, but suitable for loading.

### Methods for Preparing Immune Effector Cells

In another aspect, this disclosure also provides a method for preparing an immune effector cell expressing a CAR or TCR. The method comprises introducing into an immune effector cell a polynucleotide, a vector, a viral particle, or a virus-like particle, as described above.

In some embodiments, the method further comprises introducing into the immune effector cell (i.e., host cell) a second polynucleotide comprising a polynucleotide sequence encoding NovB2. In some embodiments, the second polynucleotide is contained in an envelope vector. In some embodiments, the envelope vector comprises an env gene is selected from VSV-G env, LCMV env, LCMV-GP(WE-HPI) env, MoMLV env, Gibbon Ape Leukemia Virus (GaLV) env; or an env gene selected from a member of the Pbabdoviridae, an Alphavirus env gene, a Paramyxovirus env gene, a Flavivirus env gene, a Retrovirus env gene, an Arenavirus env gene, a Parainfluenza virus env gene, a Thogoto virus env gene, a Baculovirus env gene, and a vesicular stomatitis virus G-protein (VSV-G) envelope vector.

In some embodiments, the immune effector cell is selected from a cytotoxic T lymphocyte (CTL), a natural killer (NK) cell, a natural killer T (NKT) cell, a tumor-infiltrating lymphocyte (TIL), a CD4T cell, a B cell, a macrophage, and a dendritic cell (DC).

In some embodiments, the method may additionally include expanding the immune effector cells in a cell culture medium following the step of introducing a polynucleotide or a vector, as described above, to the immune effector cells.

The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. The cell culture medium may include neoantigen peptides. Stable cell lines may be established to allow for the continued propagation of cells.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progenies having the same function or biological activity as screened or selected for in the originally transformed cell are included herein.

Physical methods for introducing a polynucleotide into a host cell include calcium phosphate precipitation, lipofection, particle bombardment, microinjection, electroporation, and the like. Methods for producing cells comprising exogenous vectors and/or nucleic acids are well known in the art. See, for example, Sambrook et al. (2001, Molecular Cloning: A Laboratory Manual, Cold Spring Harbor Laboratory, New York).

Chemical means for introducing a polynucleotide into a host cell include colloidal dispersion systems, such as macromolecule complexes, nanocapsules, microspheres, beads, and lipid-based systems, including oil-in-water emulsions, micelles, mixed micelles, and liposomes. An exemplary colloidal system for use as an in vitro and in vivo release vehicle is a liposome (e.g., an artificial membrane vesicle).

In the case where a non-viral delivery system is used, an exemplary delivery vehicle is a liposome. Lipid formulations can be used for the introduction of nucleic acids into a host cell (in vitro, ex vivo, or in vivo). In one example, the nucleic acid may be associated with a lipid. The nucleic acid associated with a lipid may be encapsulated in the aqueous interior of a liposome, interspersed within the lipid bilayer of a liposome, bound to a liposome via a binding molecule that is associated with both the liposome and the oligonucleotide, entrapped in a liposome, in a complex with a liposome, dispersed in a solution containing a lipid, mixed with a lipid, combined with a lipid, contained as a suspension in a lipid, content or in a complex with a micelle, or associated otherwise with a lipid. The compositions associated with lipids, lipids/DNA or lipids/expression vector are not limited to any particular structure in solution. For example, they can be present in a bilayer structure, as micelles, or with a “collapsed” structure. They can also be simply interspersed in a solution, possibly forming aggregates that are not uniform in size or shape. Lipids are fatty substances that can be natural or synthetic lipids. For example, lipids include fatty droplets that occur naturally in the cytoplasm as well as the class of compounds containing long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; Dicetylphosphate (“DCP”) can be obtained from K & K Laboratories (Plainview, NY); Cholesterol (“Choi”) can be obtained from Calbiochem-Behring; dimyristyl phosphatidylglycerol (“DMPG”) and other lipids can be obtained from Avanti Polar Lipids, Inc. (Birmingham, AL). Lipid stock solutions in chloroform or chloroform/methanol can be stored at about −20° C. Chloroform is used as the sole solvent since it evaporates more easily than methanol. “Liposome” is a generic term that encompasses a variety of unique and multilamellar lipid vehicles formed by the generation of bilayers or closed lipid aggregates. Liposomes can be characterized as having vesicular structures with a bilayer membrane of phospholipids and an internal aqueous medium. Multilamellar liposomes have multiple layers of lipids separated by an aqueous medium. They form spontaneously when phospholipids are suspended in an excess of aqueous solution. The lipid components undergo self-rearrangement before the formation of closed structures and trap dissolved water and solutes between the lipid bilayers (Ghosh et al., 1991 Glycobiology 5: 505-10). However, compositions that have different structures in solution than the normal vesicular structure are also included. For example, lipids can assume a micellar structure or simply exist as nonuniform aggregates of lipid molecules. Lipofectamine-nucleic acid complexes are also contemplated.

Regardless of the method used to introduce exogenous nucleic acids into a host cell, the presence of the recombinant DNA sequence in the host cell can be confirmed by a series of tests. Such assays include, for example, “molecular biology” assays well known to those skilled in the art, such as Southern and Northern blots, RT-PCR and PCR; biochemical assays, such as the detection of the presence or absence of a particular peptide, for example, by immunological means (ELISA and Western blot) or by assays described herein to identify agents that are within the scope of the invention.

### Methods of Treatment

This disclosure further provides a method of treating cancer or a tumor. The method comprises administering a therapeutically effective amount of a polynucleotide, a vector, a viral particle or virus-like particle, a cell, a pharmaceutical composition, as described above, or a cell prepared by a method described above to a subject in need thereof.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human.

In some embodiments, the subject is a human. In some embodiments, the subject has cancer. In some embodiments, the subject is immune-depleted.

As used to describe the present invention, “cancer,” “tumor,” and “malignancy” all relate equivalently to hyperplasia of a tissue or organ. If the tissue is a part of the lymphatic or immune system, malignant cells may include non-solid tumors of circulating cells. Malignancies of other tissues or organs may produce solid tumors. The methods of the present invention may be used in the treatment of lymphatic cells, circulating immune cells, and solid tumors.

Cancers that can be treated include tumors that are not vascularized or are not substantially vascularized, as well as vascularized tumors. Cancers may comprise non-solid tumors (such as hematologic tumors, e.g., leukemias and lymphomas) or may comprise solid tumors. The types of cancers to be treated with the compositions of the present invention include, but are not limited to, carcinoma, blastoma and sarcoma, and certain leukemias or malignant lymphoid tumors, benign and malignant tumors and malignancies, e.g., sarcomas, carcinomas, and melanomas. Also included are adult tumors/cancers and pediatric tumors/cancers.

Hematologic cancers are cancers of the blood or bone marrow. Examples of hematologic (or hematogenous) cancers include leukemias, including acute leukemias (such as acute lymphocytic leukemia, acute myelocytic leukemia, acute myelogenous leukemia, promyelocytic, myelomonocytic, monocytic, and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high-grade forms), myeloma Multiple, Waldenstrom's macroglobulinemia, heavy chain disease, myelodysplastic syndrome, hairy cell leukemia, and myelodysplasia.

Solid tumors are abnormal masses of tissue that usually do not contain cysts or liquid areas. Solid tumors can be benign or malignant. The different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma and other sarcomas, synovium, mesothelioma, Ewing tumor, leiomyosarcoma, rhabdomyosarcoma, colon carcinoma, lymphoid malignancy, pancreatic cancer, breast cancer, lung cancer, ovarian cancer, prostate cancer, hepatocellular carcinoma, squamous cell carcinoma, basal cell carcinoma, adenocarcinoma, carcinoma of the sweat gland, medullary thyroid carcinoma, papillary thyroid carcinoma, sebaceous gland carcinoma of pheochromocytomas, carcinoma papillary, papillary adenocarcinomas, medullary carcinoma, bronchogenic carcinoma, renal cell carcinoma, hepatoma, bile duct carcinoma, choriocarcinoma, Wilms tumor, cervical cancer, testicular tumor, seminoma, bladder carcinoma, melanoma, and CNS tumors (such as glioma) (such as brainstem glioma and mixed gliomas), glioblastoma (also astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastasis).

The polynucleotide, vector, viral particle or virus-like particle, cell, or pharmaceutical composition, as described, can be administered in a manner appropriate to the disease to be treated (or prevented). The amount and frequency of administration will be determined by factors such as the condition of the patient, and the type and severity of the patient's disease, although appropriate dosages can be determined by clinical trials.

When “an immunologically effective amount,” “an effective antitumor quantity,” “an effective tumor-inhibiting amount” or “therapeutic amount” is indicated, the precise amount of the compositions of the present invention to be administered can be determined by a physician having account for individual differences in age, weight, tumor size, extent of infection or metastasis, and patient's condition (subject). It can generally be stated that a pharmaceutical composition comprising the lymphocytes described herein can be administered at a dose of 10⁴ to 10⁹ cells/kg body weight, e.g., 10⁵ to 10⁶ cells/kg body weight, including all values integers within these intervals. The lymphocyte compositions can also be administered several times at these dosages. The cells can be administered using infusion techniques that are commonly known in immunotherapy (see, for example, Rosenberg et al., New Eng. J. of Med. 319: 1676, 1988). The optimal dose and treatment regimen for a particular patient can be readily determined by one skilled in the art of medicine by monitoring the patient for signs of the disease and adjusting the treatment accordingly.

The administration of the disclosed polynucleotide, vector, viral particle or virus-like particle, cell, or pharmaceutical composition can be carried out in any convenient way, including infusion or injection (i.e., intravenous, intrathecal, intramuscular, intraluminal, intratracheal, intraperitoneal, or subcutaneous), transdermal administration, or other methods known in the art. Administration can be once every two weeks, once a week, or more often, but the frequency may be decreased during a maintenance phase of the disease or disorder.

In certain cases, the cells activated and expanded using the methods described herein, or other methods known in the art wherein the cells are expanded to therapeutic levels, are administered to a patient together with (e.g., before, simultaneously, or after) any number of relevant treatment modalities. Also described herein, the cells can be used in combination with chemotherapy, radiation, immunosuppressive agents, such as cyclosporin, azathioprine, methotrexate, mycophenolate, and FK506, antibodies, or other immunoablating agents such as CAMPATH, anti-cancer antibodies. CD3 or other antibody therapies, cytoxine, fludarabine, cyclosporine, FK506, rapamycin, mycophenolic acid, steroids, FR901228, cytokines, and irradiation.

The disclosed polynucleotide, vector, viral particle or virus-like particle, cell, or pharmaceutical composition can also be administered to a patient together with (e.g., before, simultaneously or after) bone marrow transplantation, therapy with T lymphocyte ablation using chemotherapy agents such as fludarabine, radiation therapy external beam (XRT), cyclophosphamide, or antibodies such as OKT3 or CAMPATH. Also described herein, the compositions can be administered after ablative therapy of B lymphocytes, such as agents that react with CD20, for example, Rituxan. For example, subjects may undergo standard treatment with high-dose chemotherapy, followed by transplantation of peripheral blood stem cells. In certain cases, after transplantation, the subjects receive an infusion of the expanded lymphocytes, or the expanded lymphocytes are administered before or after surgery.

In some embodiments, the method may further include administering to the subject a second therapeutic agent. In some embodiments, the second therapeutic agent may be an anti-cancer or anti-tumor agent. In some embodiments, the second therapeutic agent is administered to the subject before, after, or concurrently with the vector, the viral particle or virus-like particle, the cell, or the pharmaceutical composition. In some embodiments, the second therapeutic agent may be a chemotherapeutic agent or an immunotherapeutic agent.

In some embodiments, the method further comprises administering a therapeutically effective amount of an immune checkpoint modulator. Examples of the immune checkpoint modulator may include PD1, PDL1, CTLA4, TIM3, LAG3, and TRAIL. The checkpoint modulators may be administered simultaneously, separately, or concurrently with the composition of the present invention.

A “chemotherapeutic agent” is a chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, methyldopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cyclophosphamide, estramustine, ifosfamide, mechlorethamine, mechlorethamine oxide hydrochloride, melphalan, novembichin, phentermine, prednimustine, trofosfamide, uracil mustard; nitrosoureas such as carmustine, chlorozotocin, fotemustine, lomustine, nimustine, ranimustine; antibiotics such as the enediyne antibiotics (e.g. calicheamicin, see, e.g., Agnew Chem. Intl. Ed. Engl. 33:183-186 (1994); dynemicin, including dynemicin A; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®.; razoxane; rhizoxin; sizofuran; spirogermanium; tenuazonic acid; triaziquone; 2,2′,2′″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are anti-hormonal agents that act to regulate or inhibit hormone action on tumors such as anti-estrogens, including, for example, tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, xeloda, gemcitabine, KRAS mutation covalent inhibitors and goserelin; and pharmaceutically acceptable salts, acids or derivatives of any of the above. Additional examples include irinotecan, oxaliplatinum, and other standard colon cancer regimens.

An “immunotherapeutic agent” may include a biological agent useful in the treatment of cancer. In some embodiments, the immunotherapeutic agent may include an immune checkpoint inhibitor (e.g., an inhibitor of PD-1, PD-L1, TIM-3, LAG-3, VISTA, DKG-α, B7-H3, B7-H4, TIGIT, CTLA-4, BTLA, CD160, TIM1, IDO, LAIR1, IL-12, or combinations thereof). Examples of immunotherapeutic agents include atezolizumab, avelumab, blinatumomab, daratumumab, cemiplimab, durvalumab, elotuzumab, laherparepvec, ipilimumab, nivolumab, obinutuzumab, ofatumumab, pembrolizumab, cetuximab, and talimogene.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

### ABBREVIATIONS

CAR; chimeric antigen receptor
ED; endodomain
1G, 2G, 3G, 4G; First, second, third, and fourth generation
TCR; T cell receptor
TME; tumor microenvironment
TNF-a; tumor necrosis factor alpha
IFN; interferon

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene products.” If the polynucleotide is derived from genomic DNA, expression may include splicing of the mRNA in a eukaryotic cell.

“Constitutive expression” refers to expression using a constitutive or regulated promoter. “Conditional” and “regulated expression” refer to expression controlled by a regulated promoter.

“Constitutive promoter” refers to a promoter that is able to express the open reading frame (ORF) that permits constitutive expression.

“Regulated promoter” refers to promoters that direct gene expression not constitutively, but in a temporally- and/or spatially-regulated manner, and includes both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al. (1989).

“Inducible promoter” refers to those regulated promoters that can be turned on in one or more cell types by an external stimulus, such as a chemical, light, hormone, stress, or a pathogen.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation.

As used herein, the term “recombinant” refers to a cell, microorganism, nucleic acid molecule or vector that has been modified by the introduction of an exogenous nucleic acid molecule or has controlled expression of an endogenous nucleic acid molecule or gene. Deregulated or altered to be constitutively altered, such alterations or modifications can be introduced by genetic engineering. Genetic alteration includes, for example, modification by introducing a nucleic acid molecule encoding one or more proteins or enzymes (which may include an expression control element such as a promoter), or addition, deletion, substitution of another is nucleic acid molecule, or other functional disruption of, or functional addition to, the genetic material of the cell. Exemplary modifications include modifications in the coding region of a heterologous or homologous polypeptide derived from the reference or parent molecule or a functional fragment thereof.

A peptide or polypeptide “fragment” as used herein refers to a less than full-length peptide, polypeptide or protein. For example, a peptide or polypeptide fragment can have at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length.

The term “antigen recognizing receptor,” as used herein, refers to a receptor that is capable of activating an immune cell (e.g., a T-cell) in response to antigen binding. Exemplary antigen recognizing receptors may be native or genetically engineered TCRs, or genetically engineered TCR-like mAbs (Hoydahl et al. Antibodies 2019 8:32) or CARs in which a tumor antigen-binding domain is fused to an intracellular signaling domain capable of activating an immune cell (e.g., a T-cell). T-cell clones expressing native TCRs against specific cancer antigens have been previously disclosed (Traversari et al., J Exp Med, 1992 176:1453-7; Ottaviani et al., Cancer Immunol Immunother, 2005 54:1214-20; Chaux et al., J Immunol, 1999 163:2928-36; Luiten and van der Bruggen, Tissue Antigens, 2000 55:149-52; van der Bruggen et al., Eur J Immunol, 1994 24:3038-43; Huang et al., J Immunol, 1999 162:6849-54; Ma et al., Int J Cancer, 2004 109:698-702; Ebert et al., Cancer Res, 2009 69:1046-54; Ayyoub et al. J Immunol 2002 168:1717-22; Chaux et al., European Journal of Immunology, 2001 31:1910-16; Wang et al., Cancer Immunol Immunother, 2007 56:807-18; Schultz et al., Cancer Research, 2000 60:6272-75; Cesson et al., Cancer Immunol Immunother, 2010 60:23-25; Zhang et al., Journal of Immunology, 2003 171:219-25; Gnjatic et al., PNAS, 2003 100:8862-67; Chen et al., PNAS, 2004). In one embodiment, such TCRs can be sequenced and genetically engineered into TILs for use in adoptive cell therapy. In certain aspects, TCRs that recognize MAGE-A1 antigen, MAGE-A3 antigen, MAGE A-10 antigen, MAGE-C2 antigen, NY-ESO-1 antigen, SSX2 antigen, and MAGE-A12 antigen can be genetically engineered into TILs for use in adoptive cell therapy. In yet other embodiments, genetically engineered TILs with TCRs are further engineered to secrete transgenes. In yet other embodiments, CARs are used.

“Treating” or “treatment” as used herein refers to administration of a compound or agent to a subject who has a disorder with the purpose to cure, alleviate, relieve, remedy, delay the onset of, prevent, or ameliorate the disorder, the symptom of a disorder, the disease state secondary to the disorder, or the predisposition toward the disorder.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg, etc.) usually refers to [g, mg, or other unit] “per kg (or g, mg etc.) bodyweight,” even if the term “bodyweight” is not explicitly mentioned.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, “administering” refers to the physical introduction of a composition comprising a therapeutic agent to a subject, using any of the various methods and delivery systems known to those skilled in the art. Routes of administration described herein include intravenous, intraperitoneal, intramuscular, subcutaneous, spinal or other parenteral routes of administration, for example, by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a composition described herein can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

“Parenteral” administration of composition includes, e.g., subcutaneous (s.c.), intravenous (i.v.), intramuscular (i.m.), or intrasternal injection, or infusion techniques.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent, or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the is subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%1, 1%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

This example describes the materials and methods used in EXAMPLE 2.

### Cell Lines and Culture

The prostate carcinoma cell line PC3-PIP (PMSA⁺), PC3 engineered with human CD19⁺ cells, Bjab, Na8, Me275, A375, Saos2, 293T human embryonic kidney (HEK293T) cells and Jurkat cells were cultured in RPMI-1640 supplemented with 10% heat-inactivated fetal bovine serum (FBS), 2 mmol/L L-glutamine, 100 μg/mL penicillin, and 100 U/mL streptomycin, at 37° C. in a 5% CO2 atmosphere (Invitrogen, Lifetechnologies). Na8, Me275, A375, Saos2, 293T, and Jurkat cell lines were purchased from the ATCC. The PC3-PIP and PC3 cell lines were kindly provided by Dr. A. Rosato (University of Padau, Padova, Italy) (Ghosh, A., et al. Cancer Res 65, 727-731 (2005)). Bjab was kindly provided by Dr. Caroline Arber (University of Lausanne, Switzerland). The PC3 and PC3-PIP cells lentivirally transduced to enforce expression of CD19 (PC3-CD19⁺ and PC3-PIP CD19⁺) were kindly provided by Dr. Yannick Muller (University of Lausanne, Switzerland). The HEK293T cell line was used for lentivirus vector production.

### Vector Construction

2G CARs comprising the CD8a hinge, CD28 transmembrane (TM), CD28 endodomain (ED), and CD3 zeta ED were cloned into a second-generation self-inactivating (SIN) lentiviral expression vector pELNS under the PGK promoter. The HLA-A2/NY-ESO-1₁₅₇₋₁₆₅ restricted TCR was cloned in vector pRRL, with expression also driven by the PGK promoter. The anti-PSMA scFv derived from monoclonal antibody J591 (Gong, M. C., et al. Cancer Metastasis Rev 18, 483-490 (1999)) and the anti-CD19 CAR scFv derived from monoclonal antibody FMC63 (Kochenderfer, J. N. et al. J Immunother 32, 689-702 (2009)) were used to confer tumor-antigen specificity. The HLA-A2/NY-ESO-1₁₅₇₋₁₆₅ restricted TCR has been previously described (Irving, M. et al. The Journal of Biological Chemistry 287, 23068-23078 (2012)). The (NFAT)₆ response elements-IL-2 minimal promoter; abbreviated 6xNFAT was used to evaluate inducible expression of different gene cargo. Replacement of the RSV promoter with the CMV promoter in the 5′LTR was used to enable TNFα in the culture supernatant to favor transcription of the ssRNA viral genome.

### Lentivirus Vector Production

For large scale production: briefly, 24 h before transfection, 293T cells were seeded at 10×10⁶ in 30 mL medium in a T-150 tissue culture flask. All plasmid DNA was purified using the Endo-free Maxiprep kit (Invitrogen, Life Technologies). 293T cells were transfected with 7 μg pVSVG (VSV glycoprotein expression plasmid) or 7 μg pVSVG-T2A-NovB2, 18 μg of R874 (Rev and Gag/Pol expression plasmid), and 15 μg of pELNS or pCRRL transgene plasmid, using a mix of Turbofect (Thermo Fisher Scientific AG) and Optimem media (Invitrogen, Life Technologies, 180 μL of Turbofect for 3 mL of Optimem). The cells were further transfected with a plasmid encoding the T cell leukemia virus 1, TAX protein, or the medium was further supplemented with TNFα, at 10 ng/ml working concentration. The viral supernatant was harvested 48 h post-transfection. Viral particles were concentrated by ultracentrifugation for 2 h at 24,000g and re-suspended in 400 μL complete RPMI-1640 media, followed by immediate snap freezing on dry ice.

For small scale production: briefly, 4-5 hours before transfection 293T cells were seeded at 1.25×10⁶ in 2 mL medium/well in a 6 well plate. 293T cells were transfected with 2.5 μg total DNA (divided as 0.282 μg pVSVG or pVSVG-T2A-NovB2, 0.846 μg of R874, and 1.125 μg of pELNS or pCRRL transgene plasmid, using a mix of Lipofectamine 2000 (Invitrogen) and Optimem media (Invitrogen, Life Technologies, according to manufacturer's instructions). The cells were further tranfected with a plasmid encoding the T cell leukemia virus 1, TAX protein, or the medium was further supplemented with TNFα at 10 ng/ml. The viral supernatant was harvested 48 h post-transfection and supernatant was used directly.

### Jurkat Cell Transduction for Viral Titration

Jurkat cells were suspended at 1×10⁵ cell/mL and seeded into 24-well plates at 1 mL/well. Different volumes of viral supernatant were used for transduction, as indicated, and ranging from 300 μL down to 3 μL. Cell media was refreshed after incubation for 24 h at 37° C. Viral titers (Transducing unit/mL) were calculated as follows: [(total number of cells/100)×percentage of transduced cells)×dilution of the virus supernatant].

### Primary Human T-Cell Purification, Activation, Transduction, and Expansion

Primary human T cells were isolated from the peripheral blood mononuclear cells (PBMCs) of healthy donors (HDs; prepared as buffy coats) collected with informed consent by the blood bank. Total PBMCs were obtained via Lymphoprep (Axonlab) separation solution by a standard protocol of centrifugation. CD4⁺ and CD8⁺ T cells were isolated by negative selection using magnetic beads following the manufacturer's protocol (easySEP, Stem Cell technology). Purified CD4⁺ and CD8⁺ T cells were cultured separately in RPMI-1640 with Glutamax, supplemented with 10% heat-inactivated FBS, 100 U/mL penicillin, 100 μg/mL streptomycin sulfate, and stimulated with anti-CD3 and anti-CD28 monoclonal antibody (mAb)-coated-beads (Invitrogen, Life Technologies) in a ratio of 1:2 of T cells to beads. T cells were transduced with lentivirus vector particles at 18 to 22 h post-activation. Human recombinant interleukin-2 (h-IL-2; Glaxo) was replenished every other day for a concentration of 50 IU/mL until 5d post-stimulation (day +5). At day +5, magnetic beads were removed, and h-IL7 and h-IL15 (Miltenyi Biotec GmbH) were added to the cultures at 10 ng/mL. A cell density of 0.5-1×10⁶ cells/mL was maintained for expansion. Rested engineered T cells were adjusted for equivalent transgene expression before all functional assays; the more efficiently transduced samples were diluted with appropriate numbers of untransduced (UTD) T cells

### Cytotoxicity Assays

Cytotoxicity assays were performed using The IncuCyte Instrument (Essen Bioscience). Briefly, 1.25×10⁴target cells were seeded in flat bottom 96-well plates (Costar, Vitaris). Four hours later, rested T cells (no cytokine for 48h) were washed and seeded at 2.5×10⁴/well, at a 2:1 Effector:Target (E:T) ratio in complete media. No exogenous cytokines were added during the co-culture period of the assay. CytotoxRed or Caspase-3/7green reagent (Essen Bioscience) was added at a final concentration of 125 nM in a total volume of 200 μL. Internal experimental negative controls were included in all assays, including co-incubation of untransduced (UTD) T cells and tumor cells, as well as tumor cells alone, to monitor tumor cell death over time. As a positive control, tumor cells alone were treated with 1% triton solution to evaluate maximal killing in the assay. In some assays (as indicated in the figure legends), freshly generated nuclei red and nuclei green engineered tumor cells were used. The nuclei red/green target cells were generated with IncuCyte NucLight Lentivirus (Essen Bioscience) for nuclear restricted expression of tagGFP2 (green fluorescent protein), mKate2 (red fluorescent protein), according to manufacturer's instructions. Activation of co-engineered TCR-T and CAR-T cells upon specific antigen stimulation was assessed by mCherry IncuCyte quantification over time. Images of total red area/well and green area/well were collected every 2 h of the co-culture. The total red area/well and green area/well was obtained using the analysis protocol provided by Essen Bioscience. Data were normalized by subtracting the background fluorescence observed at time 0 (i.e., before any cell killing by CAR-T cells) from all further time points. Data are expressed as the mean of different HDs±S.E.M.

### Cell Staining and Flow Cytometric Analysis

To evaluate CAR cell-surface expression, transduced cells were stained with fluorescenated anti-human F(ab′)mAb (BD Biosciences). To evaluate TCR cell surface expression, transduced cells were stained with fluorescenated HLA-A2/NY-ESO-1₁₅₇₋₁₆₅ tetramer produced in-house. Aqua live Dye BV510 and near-IR fluorescent reactive dye (APC Cy-7) were used to assess viability (Invitrogen, Life Technologies). To evaluate mCherry induction upon stimulation, T cells were stained with near-IR fluorescent reactive dye (APC Cy-7) (Invitrogen, Life Technologies). Acquisition and analysis was performed using a BD FACS LRSII and FACS DIVA software (BD Biosciences).

### Immunoblotting

Cells were lysed in RIPA buffer supplemented with Halt phosphate/protease inhibitors (Thermo Fisher Scientific) and were boiled at 97° C. for 10 minutes with Bolt LDS sample buffer and reducing agent (Thermo Fisher Scientific). Protein samples (10 g) were separated by SDS-PAGE and transferred to PVDF membranes using the iBlot2 system (Thermo Fisher Scientific). Antibody staining of the molecules of interest was carried out according to the manufacturer's instructions. Rabbit monoclonal (EP630Y) specific to MAP4K1/HPK1 antibody (ab33910) was purchased from Abcam and anti-β-actin (sc-47778) from Santa Cruz. Images were acquired with a western blot imager (Fusion, Vilber Lourmat), and protein levels were quantified using the ImageJ software by analyzing pixel intensity of the bands. Total HPK1 level was calculated by dividing its signal to the β-actin signal.

### Mouse Strain and In Vivo Experimentation

NOD scid gamma (NSG) male mice were bred and housed in a specific and opportunistic pathogen-free (SOPF) animal facility at the University of Lausanne (Epalinges, Switzerland). All in vivo experiments were conducted in accordance with and approval from the Service of Consumer and Veterinary Affairs (SCAV) of the Canton of Vaud. All cages housed 5 mice in an enriched environment providing free access to food and water. Mice were monitored at least every other day for signs of distress during experimentation and euthanized at end-point by carbon dioxide overdose.

### Subcutaneous Tumor Model and Adoptive T-Cell Transfer

NSG male mice aged 8-12-weeks were subcutaneously injected with 5×10⁶ PC3-PIP (or PC3-CD19⁺) tumor cells or 10×10⁶ Bjab. Once palpable (day 5 for PC3 and day 7 for Bjab), the mice were treated by peritumoral injection of 5×10⁶untransduced (UTD) or CAR-T cells. Tumor volume was assessed every other day by caliper measurement. Tumor volumes were calculated using the formula V=1/2(length×width²), where length is the greatest longitudinal diameter and width is the greatest transverse diameter determined via caliper measurement.

### In Vitro Bioluminescence Assay to Evaluate Inducible Gene Cargo Expression Levels for Sense Versus Antisense Lentiviral Vectors

To evaluate gene cargo expression levels for CAR- or TCR-T cells transduced with sense versus antisense lentiviral vectors comprising luciferase as the inducible gene cargo under 6xNFAT, 2.5×10⁴UTD and transduced T cells were co-cultured with target tumor cells at 1:1 E:T ratio, for 24 hours in 96 well plate. The following day the culture media was washed away and 10 μL/well of opportunely diluted Reporter Lysis 5× Buffer (Promega) was added and the cells resuspended. 50 μL/well of luciferin (PerkinElmer) was then added and cell lysate was transferred in white 96 wells white optiplate (PerkinElmer) for bioluminescence acquisition. Luciferase activity was measured by total counts acquired using the HIDEX sense 425-301i plate reader and software (Hidex).

### Proliferation Assay

To assess the proliferative capacity of A2/NY-specific TCR-T cells coexpressing a mIR-based shRNA, both transduced and UTD T cells (n=3 donors) were stained with CTV (Invitrogen, Life Technologies) according to manufacturer's instructions, prior to stimulation for 96 h with anti-CD3 and anti-CD28 monoclonal antibody (mAb)-coated-beads (Invitrogen, Life Technologies) at a 2:1 ratio of Beads:T cells, or with A2⁺/NY⁺ tumor cells lines (Me275, A375 and Saos2) and an A2⁺/NY⁻ cell line (Na8 cells) at an effector to target (E:T) ratio of 1:1.

### In Vivo Bioluminescence Imaging (BLI) Using Luciferase

Luciferase expression was evaluated in vivo from day 1 to day 11 post T-cell transfer. Mice were injected intraperitoneally with 150 mg/kg d-luciferin (PerkinElmer) in 100 μl of PBS and transferred into an anesthesia chamber induced by 3% mixture of Isofluorane and 1,5% of oxygen. Anesthetized animals were imaged at 10-35 minutes post-luciferin injection using the In-Vivo Xtreme system (In-Vivo Xtrem, Bruker Corp.) reducing anesthesia level at 1%. The photons emitted from the luciferase-expressing T cells were quantified using Molecular Imaging (MI) software (Bruker Corp.). A pseudocolor image representing the luminescence flux intensity was generated (violet and red color refer to the least and the most intense flux, respectively) then superimposed over the grayscale reference image. The luminescent region of interest was determined by drawing a gate and intensity of the signal was measured as total Photon/Second/mm/sq which correlates proportionally with the expression of Luciferase gene in transduced T cells. Mice were euthanized when the tumor volume reached 1000 mm³ according to the following formula V=1/2(length Å˜×width²), or when they met euthanasia criteria (weight loss, signs of distress) in accordance with the Swiss Federal Veterinary office and the Cantonal Veterinary Office guidelines.

### Statistical Analysis

GraphPad Prism 9.0 analysis software was used to determine statistically significant differences using one-way ANOVA followed by Tukey post-hoc correction for multiple comparison analysis (column groups, one variable tested). A two-way repeated measurement ANOVA followed by Tukey post-hoc correction test was used for statistical analysis of tumour growth curves, in vitro cytotoxicity, and mCherry induction analysis (two variables analysis for multiple groups). Differences were considered significant when *p<0.05, very significant when **p<0.01, and highly significant when ***p<0.001. “ns” stands for non-significant.

TABLE 3
Representative sequences of miR

| | | SEQ |
|---|---|---|
| | | ID |
| miR ID | miR Seq | NO |
| miR Ctrl | TTGAATGAGGCTTCAGTACTTTACAGAATCGTTGCCTGCACATC | 1 |
| | TTGGAAACACTTGCTGGGATTACTTCTTCAGGTTAACCCAACAG | |
| | AAGGCTAAAGAAGGTATATTGCTGTTGACAGTGAGCGaaggcagaa | |
| | gtatgcaaagcatTAGTGAAGCCACAGATGTAatgctttgcatacttctgcctgTGCC | |
| | TACTGCCTCGGACTTCAAGGGGCTACTTTAGGAGCAATTATCTT | |
| | GTTTACTAAAACTGAATACCTTGCTATCTCTTTGATACATTTTTA | |
| | CAAAGCTGAATTAAAATGGTATAAATTAAATCACTTT | |
| miR | TTGAATGAGGCTTCAGTACTTTACAGAATCGTTGCCTGCACATC | 2 |
| MAP4K1A | TTGGAAACACTTGCTGGGATTACTTCTTCAGGTTAACCCAACAG | |
| | AAGGCTAAAGAAGGTATATTGCTGTTGACAGTGAGCGAAGCTA | |
| | ACATCCTCATCAATGATAGTGAAGCCACAGATGTATCATTGAT | |
| | GAGGATGTTAGCTCTGCCTACTGCCTCGGACTTCAAGGGGCTA | |
| | CTTTAGGAGCAATTATCTTGTTTACTAAAACTGAATACCTTGCT | |
| | ATCTCTTTGATACATTTTTACAAAGCTGAATTAAAATGGTATAA | |
| | ATTAAATCACTTT | |
| miR | TTGAATGAGGCTTCAGTACTTTACAGAATCGTTGCCTGCACATC | 3 |
| MAP4K1B | TTGGAAACACTTGCTGGGATTACTTCTTCAGGTTAACCCAACAG | |
| | AAGGCTAAAGAAGGTATATTGCTGTTGACAGTGAGCGACCCCA | |
| | AGAAGGAAAAGATGAATAGTGAAGCCACAGATGTATTCATCTT | |
| | TTCCTTCTTGGGGGTGCCTACTGCCTCGGACTTCAAGGGGCTAC | |
| | TTTAGGAGCAATTATCTTGTTTACTAAAACTGAATACCTTGCTA | |
| | TCTCTTTGATACATTTTTACAAAGCTGAATTAAAATGGTATAAA | |
| | TTAAATCACTTT | |
| miR | TTGAATGAGGCTTCAGTACTTTACAGAATCGTTGCCTGCACATC | 4 |
| MAP4K1C | TTGGAAACACTTGCTGGGATTACTTCTTCAGGTTAACCCAACAG | |
| | AAGGCTAAAGAAGGTATATTGCTGTTGACAGTGAGCGATGCCC | |
| | TTCTCGTAAAGTTGTATAGTGAAGCCACAGATGTATACAACTTT | |
| | ACGAGAAGGGCACTGCCTACTGCCTCGGACTTCAAGGGGCTAC | |
| | TTTAGGAGCAATTATCTTGTTTACTAAAACTGAATACCTTGCTA | |
| | TCTCTTTGATACATTTTTACAAAGCTGAATTAAAATGGTATAAA | |
| | TTAAATCACTTT | |

TABLE 4
Representative sequences of components encoded in lentiviral vectors

| | | SEQ |
|---|---|---|
| Vector | | ID |
| Component | Sequence | NO |
| (NFAT)₆ | GGAGGAAAAACTGTTTCATACAGAAGGCGTGGAGGAAAAA | 5 |
| response | CTGTTTCATACAGAAGGCGTGGAGGAAAAACTGTTTCATAC | |
| elements-IL- | AGAAGGCGTGGAGGAAAAACTGTTTCATACAGAAGGCGTG | |
| 2 minimal | GAGGAAAAACTGTTTCATACAGAAGGCGTGGAGGAAAAAC | |
| promoter | TGTTTCATACAGAAGGCGTGACATTTTGACACCCCCATAAT | |
| (abbreviated | ATTTTTCCAGAATTAACAGTATAAATTGCATCTCTTGTTCAA | |
| 6xNFAT) | GAGTTCCCTATCACTCTCTTTAATCACTACTCACAGTAACCT | |
| | CAACTCCTGC | |
| BGH | CTGTGCCTTCTAGTTGCCAGCCATCTGTTGTTTGCCCCTCCC | 6 |
| Polyadenyla- | CCGTGCCTTCCTTGACCCTGGAAGGTGCCACTCCCACTGTCC | |
| tion sequence | TTTCCTAATAAAATGAGGAAATTGCATCGCATTGTCTGAGT | |
| | AGGTGTCATTCTATTCTGGGGGGTGGGGTGGGGCAGGACAG | |
| | CAAGGGGGAGGATTGGGAAGACAATAGCAGGCATGCTGGG | |
| | GATGCGGTGGGCTCTATGG | |
| SPA + | CTAGCAATAAAAGATCTTTATTTTCATTAGATCTGTGTGTTG | 7 |
| human | GTTTTTTGTGTGTCTAGTTAATTAAGGTGGGGCAGTGGGGG | |
| transcr. | CCAACTTGTCCTTACCCAGAGTGCAGGTGTGTGGAGATCCC | |
| pausing site | TCCTGCCTTGACATTGAGCAGCCTTAGAGGGTGGGGGAGGC | |
| | TCAGGGGTCAGGTCTCTGTTCCTGCTTATTGGGGAGTTCCTG | |
| | GCCTGGCCCTTCTATGTCTCCCCAGGTACCCCAGTTTTTCTG | |
| | GGTTCACCCAGAGTGCAGATGCTTGAGGAGGTGGGAAGGG | |
| | ACTATTTGGGGGTGTCTGGCTCAGGTGCCATGCCTCACTGG | |
| | GGCTGGTTGGCACCTGCATTTCCTGGGAGTGGGGCTGTCTC | |
| | AGGGTAGCTGGGCACGGTGTTCCCTTGAGTGGGGGTGTAGT | |
| | GGGTGTTCCTAGCTGCCACGCCTTTGCCTTCACCTATGGGAT | |
| | CGTGGCTGTCAG | |
| TAX Protein | ATGGCACACTTTCCTGGGTTTGGGCAGTCACTGCTCTTTGGG | 8 |
| | TACCCCGTTTATGTATTCGGCGACTGTGTTCAAGGAGACTG | |
| | GTGTCCTATCTCAGGCGGTCTCTGTTCCGCTCGACTCCACCG | |
| | GCATGCACTCCTCGCGACATGTCCCGAGCATCAGATAACCT | |
| | GGGATCCAATCGACGGAAGGGTGATAGGCTCTGCCCTGCAG | |
| | TTTCTCATCCCTCGCCTGCCCAGCTTTCCCACCCAGCGAACC | |
| | TCCAAAACCTTGAAGGTCTTGACCCCTCCAATCACACATAC | |
| | TACCCCTAACATCCCGCCTTCCTTCCTGCAAGCCATGCGAAA | |
| | ATACAGCCCCTTCAGAAACGGTTACATGGAACCTACTTTGG | |
| | GTCAGCACCTGCCTACATTGTCCTTTCCGGACCCCGGCCTGC | |
| | GACCGCAGAACCTGTATACACTGTGGGGCGGCTCTGTCGTC | |
| | TGTATGTACCTCTACCAGCTGTCACCGCCCATCACGTGGCCC | |
| | CTGTTGCCCCATGTAATATTTTGCCACCCAGGACAGCTCGGC | |
| | GCCTTTCTGACAAACGTCCCTTATAAACGAATCGAAAAACT | |
| | CCTGTATAAAATTTCACTGACCACCGGAGCCCTGATAATAC | |
| | TGCCTGAAGACTGCCTGCCAACCACTCTGTTTCAACCCGCTA | |
| | GGGCTCCCGTCACCCTCACTGCTTGGCAGAACGGACTCCTG | |
| | CCCTTCCATTCCACCCTCACAACTCCAGGGTTGATTTGGACA | |
| | TTTACCGATGGAACTCCTATGATTTCCGGACCTTGTCCCAAG | |
| | GACGGACAGCCATCCTTGGTGCTCCAATCCTCCTCCTTTATT | |
| | TTCCACAAGTTTCAAACGAAGGCATACCATCCTAGCTTCCT | |
| | GCTGAGCCATGGACTGATACAGTACTCTTCTTTTCATAATCT | |
| | GCACCTGTTGTTTGAGGAGTATACTAATATCCCAATCAGCCT | |
| | CCTCTTTAATGAAAAAGAGGCGGACGACAATGATCATGAGC | |
| | CGCAGATCTCACCTGGCGGGCTGGAGCCCCTTAGCGAAAAG | |
| | CATTTTCGCGAAACAGAGGTC | |
| U6 | GAGGGCCTATTTCCCATGATTCCTTCATATTTGCATATACGA | 9 |
| | TACAAGGCTGTTAGAGAGATAATTGGAATTAATTTGACTGT | |
| | AAACACAAAGATATTAGTACAAAATACGTGACGTAGAAAG | |
| | TAATAATTTCTTGGGTAGTTTGCAGTTTTAAAATTATGTTTT | |
| | AAAATGGACTATCATATGCTTACCGTAACTTGAAAGTATTT | |
| | CGATTTCTTGGCTTTATATATCTTGTGGAAAGGAC | |
| SFFV | GTAACGCCATTTTGCAAGGCATGGAAAAATACCAAACCAAG | 10 |
| | AATAGAGAAGTTCAGATCAAGGGCGGGTACATGAAAATAG | |
| | CTAACGTTGGGCCAAACAGGATATCTGCGGTGAGCAGTTTC | |
| | GGCCCCGGCCCGGGGCCAAGAACAGATGGTCACCGCAGTTT | |
| | CGGCCCCGGCCCGAGGCCAAGAACAGATGGTCCCCAGATAT | |
| | GGCCCAACCCTCAGCAGTTTCTTAAGACCCATCAGATGTTTC | |
| | CAGGCTCCCCCAAGGACCTGAAATGACCCTGCGCCTTATTT | |
| | GAATTAACCAATCAGCCTGCTTCTCGCTTCTGTTCGCGCGCT | |
| | TCTGCTTCCCGAGCTCTATAAAAGAGCTCACAACCCCTCACT | |
| | CGGCGCGCCAGTCCTCCGACAGACTGAGTCGGCCGG | |
| NovB2 | ATGACCAACATGAGCTGCGCCTACGAGCTGATCAAGAGCCT | 11 |
| | GCCCGCCAAGCTGGAACAGCTGGCCCAGGAAACCCAGGCC | |
| | ACCATCCAGACCCTGATGATCGCCGACCCCAACGTGAACAA | |
| | GGACCTGCGGGCCTTCTGCGAGTTCCTGACCGTGCAGCACC | |
| | AGCGGGCCTACAGAGCCACCAACAGCCTGCTGATTAAGCCC | |
| | AGAGTGGCCGCTGCCCTGAGAGGCGAGGAACTGGATCTGG | |
| | GAGAGGCCGATGTGGCCGCTAGAGTGCGGCAGCTGAAACA | |
| | GCAGCTGGCCGAGCTGGAAATGGAAATCAAGCCCGGCCAT | |
| | CAGCAGGTGGCCCAGGTGTCCGGAAGAAGAAAAGCCGCCG | |
| | CTGCCGCTCCTGTGGCTCAGCTGGGAAGAGTGGGAGTCGTG | |
| | AACGAG | |
| CMV-5′LTR | GTGATGCGGTTTTGGCAGTACATCAATGGGCGTGGATAGCG | 12 |
| promoter | GTTTGACTCACGGGGATTTCCAAGTCTCCACCCCATTGACGT | |
| | CAATGGGAGTTTGTTTTGGCACCAAAATCAACGGGACTTTC | |
| | CAAAATGTCGTAACAACTCCGCCCCATTGACGCAAATGGGC | |
| | GGTAGGCGTGTACGGTGGGAGGTCTATATAAGCAGAGCT | |

### Example 2

### Antisense Vector Design to Accommodate Independent Promoters

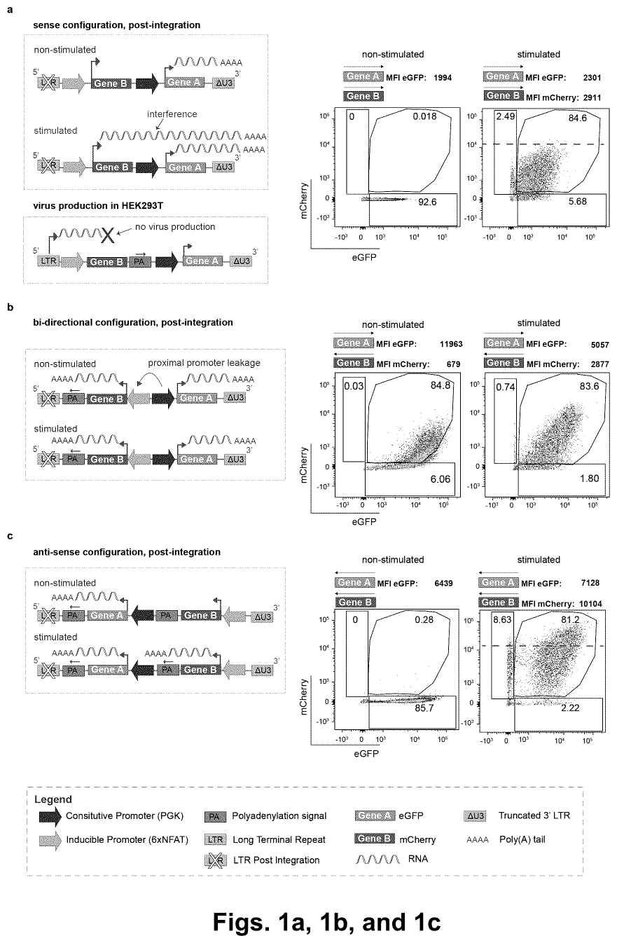

In this example, it was sought to optimize lentivirus vector-mediated, independent coexpression of two genes in transduced human T cells, with one gene under a constitutive promoter and the other under an inducible promoter, to improve adoptive T cell transfer (ACT) of cancer. A panel of transfer vectors was constructed, with the promoters in dual sense and bidirectional orientations (FIGS. 1a and 1b, left). As an example, the constitutive human phosphoglycerate kinase (PGK) promoter for gene A and 6xNFAT for gene B were tested. For screening purposes, eGFP was placed under the control of PGK, and mCherry was placed under the control of 6xNFAT (lentivirus vector component sequences are found in Table 4).

The production of second generation lentivirus vectors relies upon the co-transfection of: (i) a transfer, (ii) a packaging, and (iii) an envelope vector into a producer cell line like human embryonic kidney (HEK)293T cells (i.e., HEK293 cells expressing the oncogenic SV40 large T-antigen thought to promote plasmid-mediated gene expression) (Merten, O W., et al. Mol Ther Methods Clin Dev 3, 16017 (2016)). Lentiviral vectors typically comprise three HIV-1 genes: (i) gag (which is processed to a matrix and other retroviral core proteins), (ii) pol (reverse transcriptase, RNase H, and integrase functions), both found on the packaging plasmid, and (iii) env (envelope protein that resides in the lipid bilayer and determines viral tropism) on the envelope vector. The vesicular stomatitis virus G-protein (VSV-G) pseudotype was used, which broadens the type of cells that can be infected as compared to the HIV envelope. Notably, the transfer vector does not encode viral sequences, except for necessary cis-acting sequences such as the long terminal repeat (LTR), packaging signals, and the Woodchuck hepatitis virus post-transcriptional regulatory element (WPRE) to enhance expression of the transgene. The LTRs, located at each end of the provirus, comprise U3, R, and U5 regions, and function as a eukaryotic transcription unit. Specifically, the U3 region contains the viral promoter and enhancer elements, the R region includes the mRNA initiation site, and the U5 region is involved with polyadenylation. Notably, the 3′LTR of the transfer vector has been truncated (U3 has been removed) to generate self-inactivating lentivirus vectors (SIN).

Here, to produce lentiviral particles, HEK293T cells were transfected with lentiviral packaging and envelope plasmids, along with differently designed transfer vectors, and crude supernatant was used directly to transduce Jurkat cells. For the sense transfer vector configuration, the 6xNFAT promoter and gene B (mCherry) were placed in the same orientation upstream of the PGK promoter and gene A (eGFP) (FIG. 1a, top left). Indeed, the inducible promoter cannot be placed downstream of the constitutive one as there will be readthrough, and hence constitutive expression, of both genes by the upstream promoter. Moreover, it is not possible to place a polyadenylation (PA) site between the two genes to avoid interference because this will abrogate virus production in the HEK293T cells (depicted in FIG. 1a, bottom left).

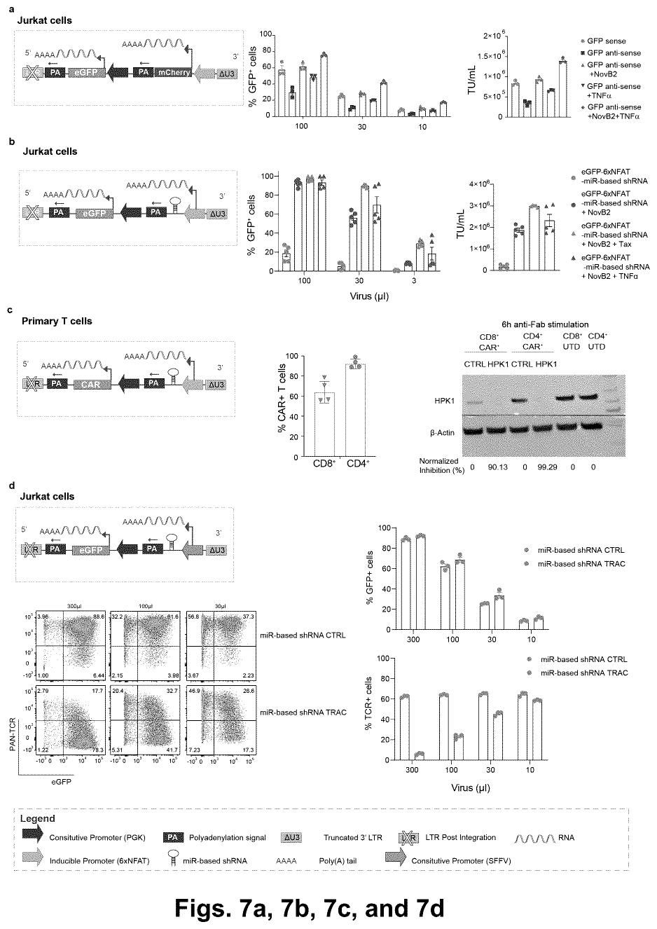

Expression of dual sense orientation genes, as described above, was evaluated in unstimulated and stimulated Jurkat cells. Expression of eGFP in unstimulated Jurkat cells, and co-expression of both eGFP and mCherry upon stimulation (FIG. 1a, right) were observed. For the latter, transcription of both genes must reach the same 3′ long terminal repeat (LTR) for polyadenylation to occur, and it has been previously reported that this configuration can cause transcriptional interference, which is limiting to transgene expression. Indeed, interference resulting from the dual sense configuration is evident upon a comparison of the Mean Fluorescence Intensity (MFI) for mCherry when encoded alone versus upstream of constitutively expressed GFP (FIG. 7a).

To avoid such interference, a bidirectional configuration (FIG. 1b, left) in which the orientation of Gene B and its promoter are inverted was next evaluated. Notably, for inverted Gene B, no longer restricted by polyadenylation at the LTR, an inverted bovine growth hormone (BGH) PA site was employed. Of note, an inverted PA site did not interfere with virus production. However, despite the separation of the two gene cassettes, leakage from the inducible promoter was observed, as evidenced by mCherry expression in non-activated Jurkat cells, presumably due to the proximity of strong enhancer elements of the constitutive promoter (FIG. 1b, right).

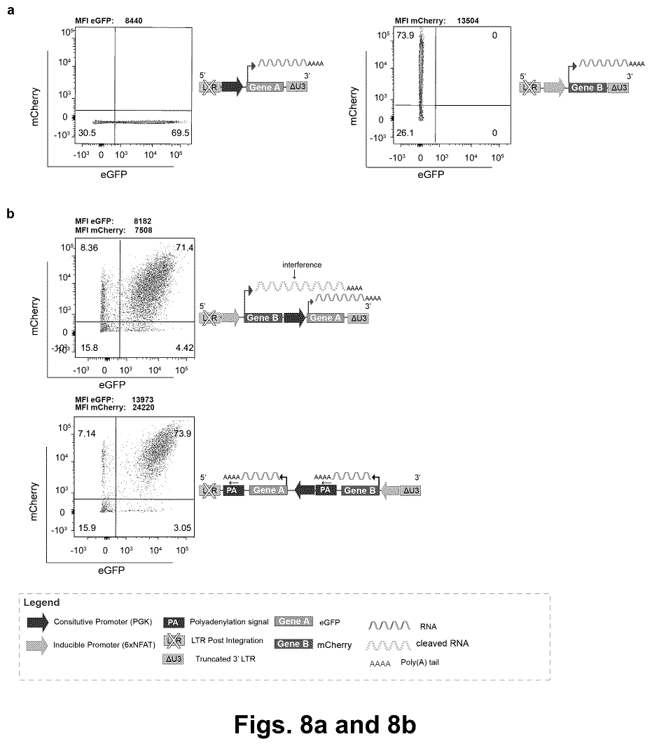

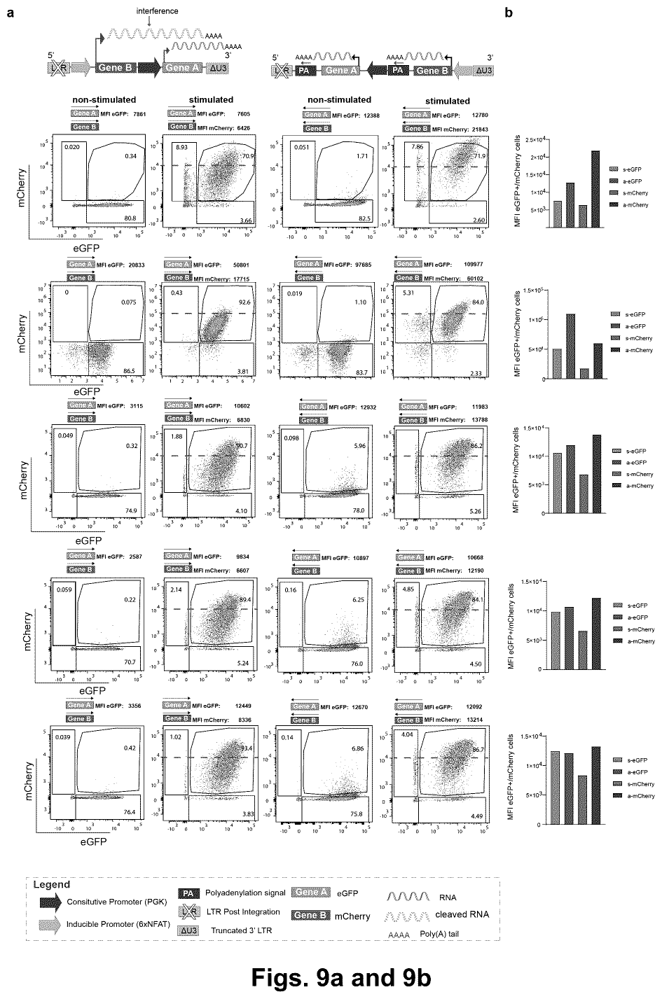

Finally, in order to prevent both interference and leakage issues, as seen for the first two transfer vector designs, a dual antisense configuration vector was constructed (FIG. 1c, left), in which Gene A has its own PA signal derived from BGH, and Gene B is followed by a synthetic polyadenylation site (SPA) and a human transcription pausing site (to prevent transcriptional read-through). The highest level of expression of both eGFP and mCherry in activated Jurkat cells amongst the 3 configurations evaluated was observed, and there was no mCherry expressed in nonactivated Jurkat cells. For example, in the representative experiment shown in FIG. 1, in stimulated Jurkat cells, an MFI for mCherry of 10104 was observed for the antisense configuration (FIG. 1c, right) versus an MFI of 2911 for the sense configuration vector (FIG. 1a, right). While absolute MFI values varied between independent assays, within a given experiment, a higher MFI for both eGFP and mCherry in activated Jurkat cells transduced with the dual antisense in comparison to dual sense lentiviral vector was consistently observed (FIG. 8 and FIG. 9). This is likely due to the lack of transcriptional interference as well as the use of the BGH PA site which is stronger than polyadenylation by the LTR. The study was thus continued with this dual inverted transfer vector configuration.

### Overcoming Low Lentiviral Titers: Abrogating the Anti-dsRNA Response

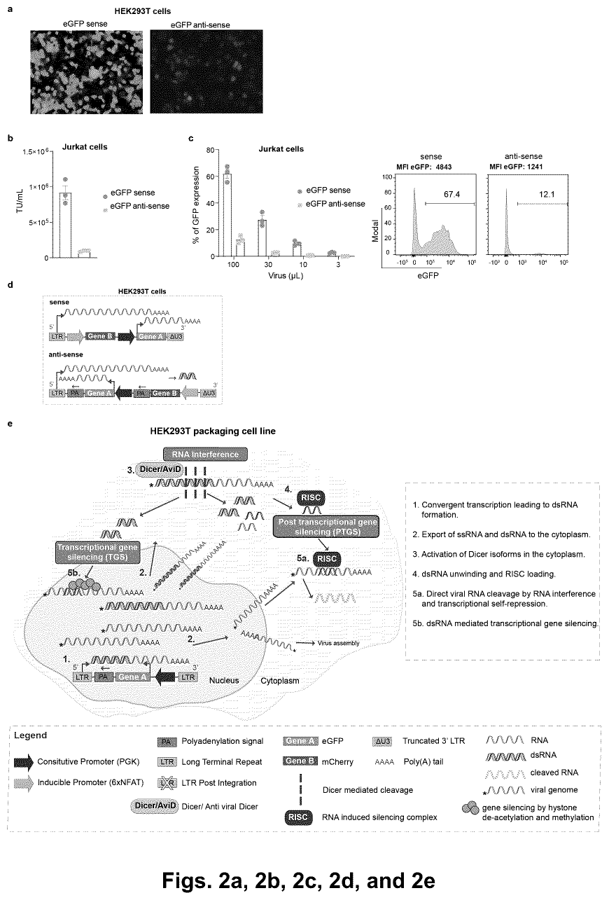

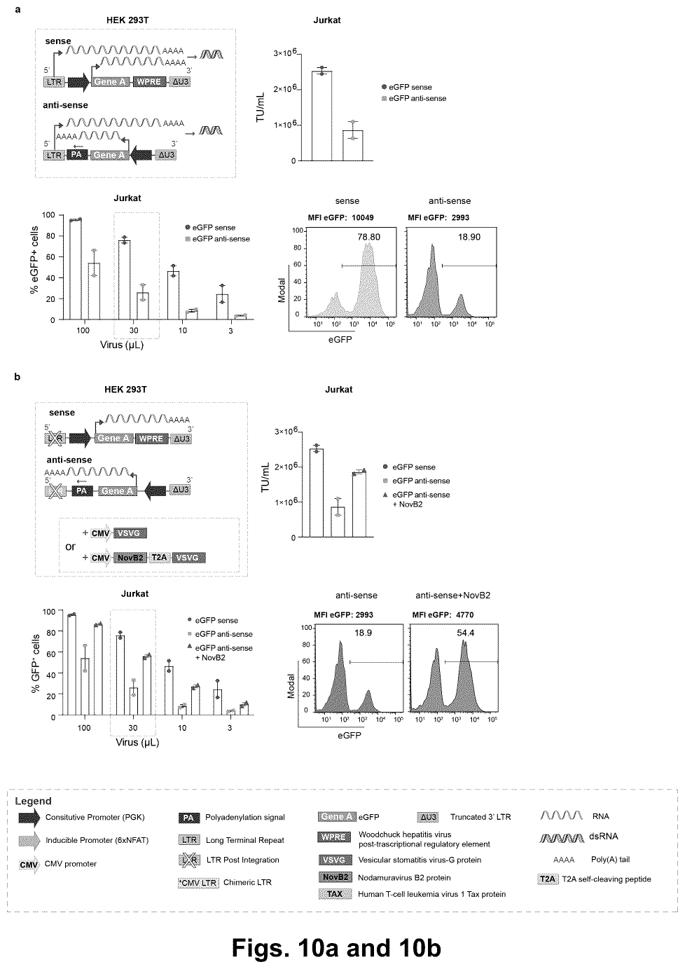

Post-integration, the dual antisense vector configuration enabled the best co-expression of both a constitutive and an inducible gene in transduced, activated Jurkat cells (i.e., no competition to reach the PA site, no leakiness by the inducible promoter, and highest MFI of both eGFP and mCherry post-activation) (FIG. 1, FIG. 8, and FIG. 9). However, during lentivirus vector production, there was an obvious decrease in eGFP expression levels for vectors comprising the dual antisense versus sense orientation of the transgenes (FIG. 2a), which corresponded to much lower viral titers for the antisense lentiviral vectors (FIG. 2b). Indeed, transduction of Jurkat cells with 100 μL lentiviral supernatant yielded about 60% transduction efficiency for the dual sense orientation vector versus about 10% (and lower MFI) for the dual inverted vector (FIG. 2c). Similarly, for single gene-cassettes, lower viral titers were observed for anti-sense versus sense lentiviral vectors (FIG. 10a).

Hence, it was next sought to overcome barriers to the production of lentiviral particles comprising an anti-sense transfer vector. During lentivirus vector production in HEK293T cells, both the 5′LTR and the inverted PKG promoter of the antisense vector are active, thus resulting in the generation of double-stranded (ds)RNA by convergent transcription (FIG. 2d). Although intracellular innate immunity may be triggered in response to dsRNA upon detection by nuclear and cytosolic sensors such as during a natural viral infection, this has been shown not to limit lentivirus vector titer because HEK293T do not generate an interferon (IFN) response. Indeed, it has recently been revealed that HEK293T as well as various stem cell-like lines employ an RNA interference (RNAi) response involving various Dicer isoforms upon detection of dsRNA. It was thus postulated that the dsRNA resulting from convergent transcription may be subject to Dicer and/or Dicer isoform-mediated (e.g., aviD) cleavage within the nucleus or cytoplasm and that small siRNA products created during this process are involved either in RNAi mediated self-degradation of the viral RNA to be packaged, or/and in transcriptional gene silencing of the viral RNA to be packaged (FIG. 2e).

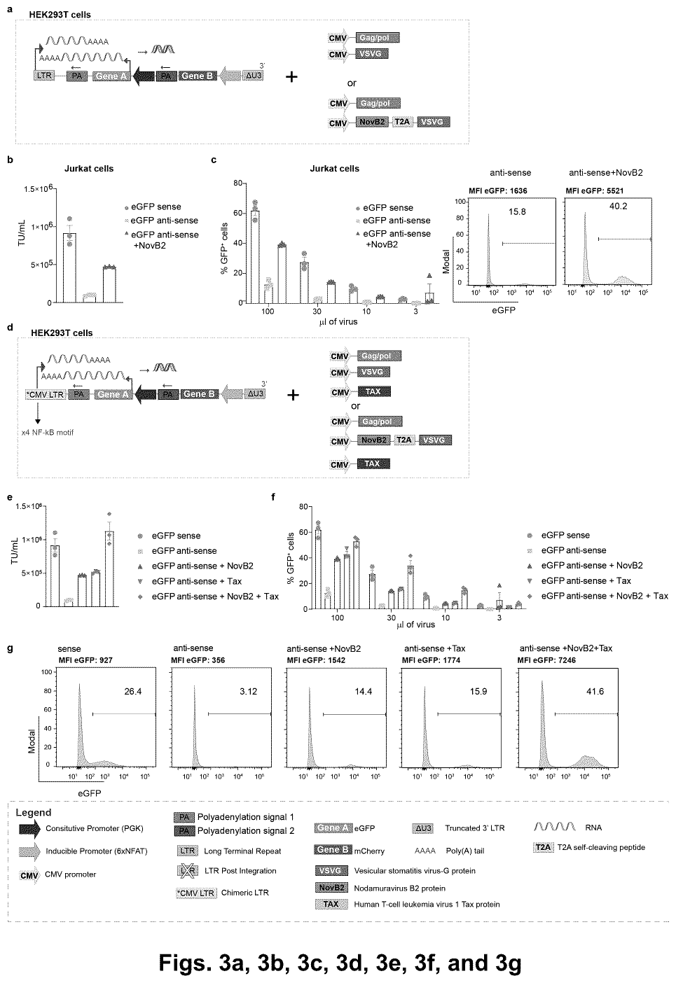

Two approaches were devised to overcome these potential barriers to lentivirus vector production arising from convergent transcription. The first approach is to inhibit antiviral RNAi machinery so as to prevent disruption of the viral genome by taking advantage of a natural viral mechanism to evade immunity. Specifically, Nodamuravirus expresses an RNA interference suppressor protein called B2 (hereafter referred to as NovB2) (Poirier, E. Z. et al. Science 373, 231-236 (2021); Sullivan, C. S. & Ganem, D. J Virol 79, 7371-7379 (2005)), and NovB2 has been previously utilized to increase viral titers of bidirectional vectors by at least five-fold via inhibition of Dicer isoforms. The strategy of co-expressing NovB2 from the envelope vector was hence employed (FIG. 3a). As a result, a significant increase in viral titer was achieved (FIG. 3b). Indeed, a five-fold rise in the proportion of eGFP⁺ Jurkat cells upon transduction with dual antisense lentivirus vector was observed (FIG. 3c). The use of NovB2 also increased titers for single gene-cassette inverted lentiviral vectors (FIG. 10b).

### Overcoming Low Lentiviral Titers: Favoring Transcription of the Viral Genome

The second approach is to favor the transcription of the viral genome for packaging (i.e., ssRNA transcription from the 5′LTR) by exploiting the Human T-cell leukemia virus 1 Tax protein. The Tax protein (Suzuki, N. et al. Sci Rep 8, 15036 (2018)) is associated with the transcriptional promotion of viral proteins (including in the nucleus during infection) and the regulation of many signaling pathways, including CREB/ATF, NF-κB, AP-1, and RSF. To test if Tax could be used to increase viral titers, the initial RSV-based promoter and enhancer region at the 5′LTR were replaced with the cytomegalovirus (CMV) promoter and enhancer, which comprises 4 consensus NF-κB binding motifs (the schematic shown in FIG. 3d). Virus was then produced in the presence or absence of co-transfected Tax-expressing plasmid (FIG. 3d). A similar gain was observed in titer, transduction efficiency and transgene expression levels (MFI) as achieved in the context of NovB2 (FIGS. 3e-3g). It is likely that the Tax-mediated increase in lentivirus vector titer was due to a change of stoichiometry in favor of viral genome transcript and higher transcription of the packaging and envelope vectors having CMV promoters. Finally, it was found that Tax and NovB2 were able to act jointly to restore antisense viral titers, transduction efficiency, and levels of transgene expression (MFI) (FIGS. 3e-3g)

### Inducible Gene Cargo Encoded in Antisense is Efficiently Expressed Upon T Cell Activation In Vitro

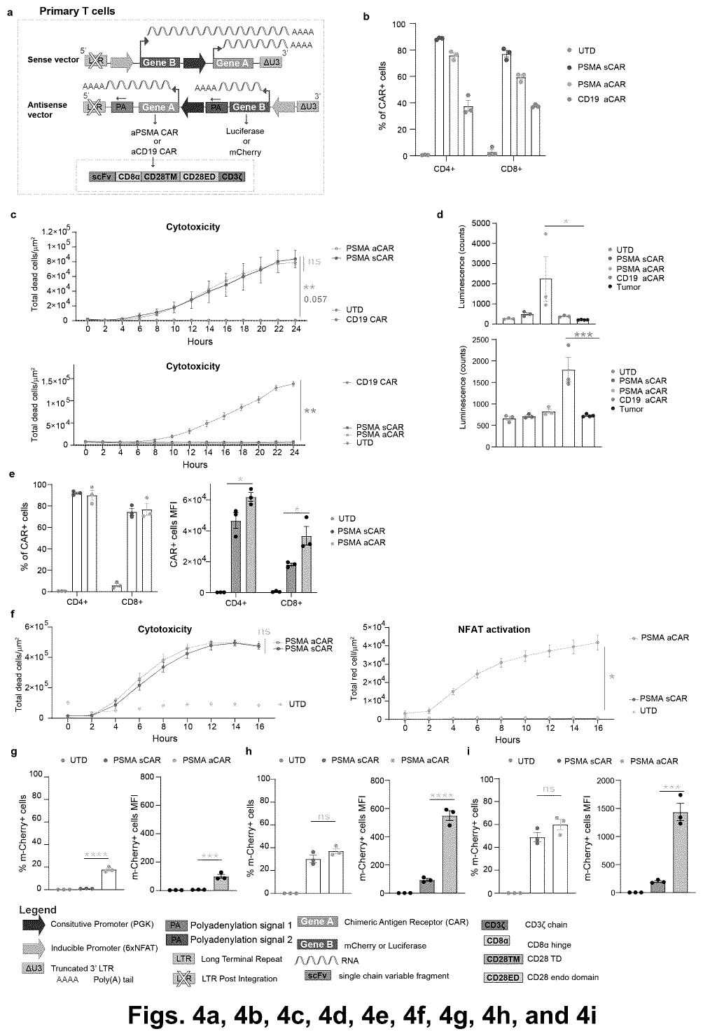

Next, the dual inverted vector design and optimized methodology for lentivirus vector production was tested in the context of both next generation (4G) CAR- and TCR-T cells. For proof-of-principle, vectors comprising an anti-PSMA or anti-CD19 CAR (constitutively expressed under PGK) (Giordano-Attianese, G. et al., Nat Biotechnol (2020)) were constructed, along with luciferase as inducible gene cargo (under 6xNFAT) (FIG. 4a). An equivalent sense orientation transfer vector for the anti-PSMA CAR and luciferase was also generated. A lentivirus vector was produced in the presence of NovB2 and Tax, and it was observed that both human CD4⁺ and CD8⁺ T cells were efficiently transduced with the 4G constructs (FIG. 4b). To achieve an equivalent percentage of 4G CAR⁺ T cells for functional testing, the transduced T cells were mixed with untransduced (UTD) T cells to reach 40% CAR⁺ (i.e., the lowest transduction efficiency as achieved for CD8⁺ T cells with the 4G anti-CD19 CAR, FIG. 4b). The 4G CAR-T cells all efficiently and specifically killed target cells in co-culture assays (FIG. 4c, upper and bottom panels). While there were no differences in specific target cell killing by the 4G anti-PSMA CAR-T cells generated with sense versus antisense lentiviral vectors, significantly higher levels of luciferase mediated luminescence were observed for the anti-sense design (FIG. 4d).

Sense and antisense lentiviral transfer vectors encoding the anti-PSMA CAR and mCherry as inducible gene cargo were further compared. Once again, lentivirus vector was produced in the presence of NovB2 and Tax, and efficient transduction of both human CD4⁺ and CD8⁺ T cells was achieved (FIG. 4e, left). A significantly higher MFI for CARs expressed from the dual antisense versus sense lentiviral vectors was further observed (FIG. 4e, right). In line with the findings above, no differences were observed in cytotoxicity of target PC3-PIP tumor cells by anti-PSMA CAR-T cells generated with the different orientation lentiviral vectors (FIG. 4f, left). It is possible that more stringent conditions, such as the use of a weaker CAR, the co-culture of fewer CAR-T cells to target cells, or the use of tumor cells with lower levels of the target antigen, may reveal lower relative activity levels of CAR-T cells generated with the sense lentiviral vector. Upon T-cell activation in co-culture assays, mCherry expression levels steadily increased over time for the antisense lentiviral vector generated 4G CAR-T cells, but mCherry was not detectable for the sense lentiviral vector engineered CAR-T cells, even at 16 h (FIG. 4f, right). To confirm that this lack of detection was a sensitivity issue for the IncuCyte instrument-based assay, rather than a defect in the sense vector, the 4G CAR-T cells following 24 h coculture without and with target cells by flow cytometric analysis were evaluated. Higher background levels of mCherry expression were observed for the antisense vector (both percentage and MFI) in non-activated 4G CAR-T cells (FIG. 4g). However, similar transduction efficiencies were achieved for both the sense and antisense vectors as evidenced by the percentage of T cells expressing mCherry upon T-cell activation (i.e., the sense orientation lentiviral vector is functional; FIG. 4h, left) and the MFI for mCherry upon activation was significantly higher for the antisense lentiviral vector (FIG. 4h, right). Significantly higher levels of mCherry expression (MFI) were also observed upon phorbol myristate acetate (PMA)-Ionomycin stimulation of the antisense lentiviral vector generated 4G CAR-T cells (FIG. 4i).

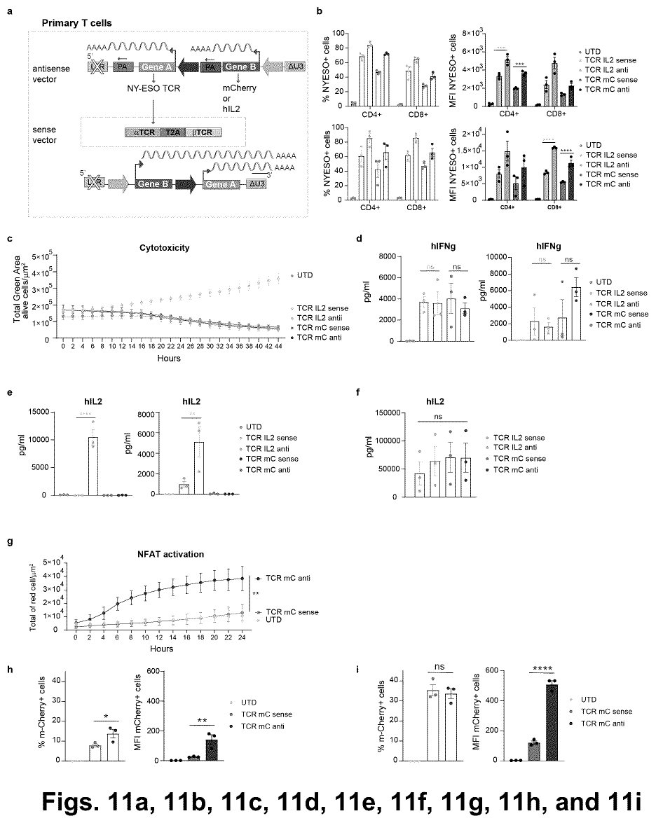

Subsequently, lentiviral transfer vectors were developed, encoding a clinically relevant HLA-A2 restricted NY-ESO-1₁₅₇₋₁₆₅ specific TCR (Irving, M. et al. The Journal of Biological Chemistry 287, 23068-23078 (2012)) along with either IL-2 or mCherry as inducible gene cargo (FIG. 11a). Lentivirus vector encoding the TCR and IL-2 was produced in the presence of NovB2 and Tax, and human CD4⁺ and CD8⁺ T cells were efficiently transduced (FIG. 11b). As for the CAR-T cells, equivalent percentages of TCR⁺-T cells were generated by appropriate mixing with UTD-T cells for all comparative functional assays. Similar levels of target cell killing (FIG. 11c) were observed as well as IFNg production (FIG. 11d) upon coculture with A2⁺/NY⁺ Saos-2 target cells for the IL-2 co-engineered TCR-T cells generated either with sense or antisense vectors. However, significantly higher levels of IL-2 were produced by the next-generation TCR-T cells generated with the antisense versus sense vector upon coculture with target cells (FIG. 11e). Differences in IL-2 gene cargo expression levels were not observed upon PMA-Ionomycin stimulation of the engineered T cells, a condition that drives the maximum production of endogenous IL-2 (FIG. 11f). Finally, TCR-T cells with inducible mCherry as gene cargo generated from antisense versus sense vectors were tested. Upon T-cell activation in co-culture assays with target cells, an increase in mCherry was evident over time for the antisense but not for the sense lentiviral vector generated TCR-T cells (FIG. 11g). However, flow cytometric analysis of next-generation TCR-T cells following 24 h coculture with target A2⁺/NY⁺ Saos-2 target tumor cells confirmed that mCherry was in fact produced by T cells generated with both antisense and sense lentiviral vectors (FIG. 11h, left) but that mCherry expression levels (MFI) were very low for the sense orientation (FIG. 11h, right). Significantly higher levels of mCherry expression (MFI) were also observed upon PMA-Ionomycin stimulation of the antisense versus sense lentiviral vector generated TCR-T cells (FIG. 11i).

### Inducible Gene Cargo Encoded in Antisense is Efficiently Expressed Upon T Cell Activation In Vivo

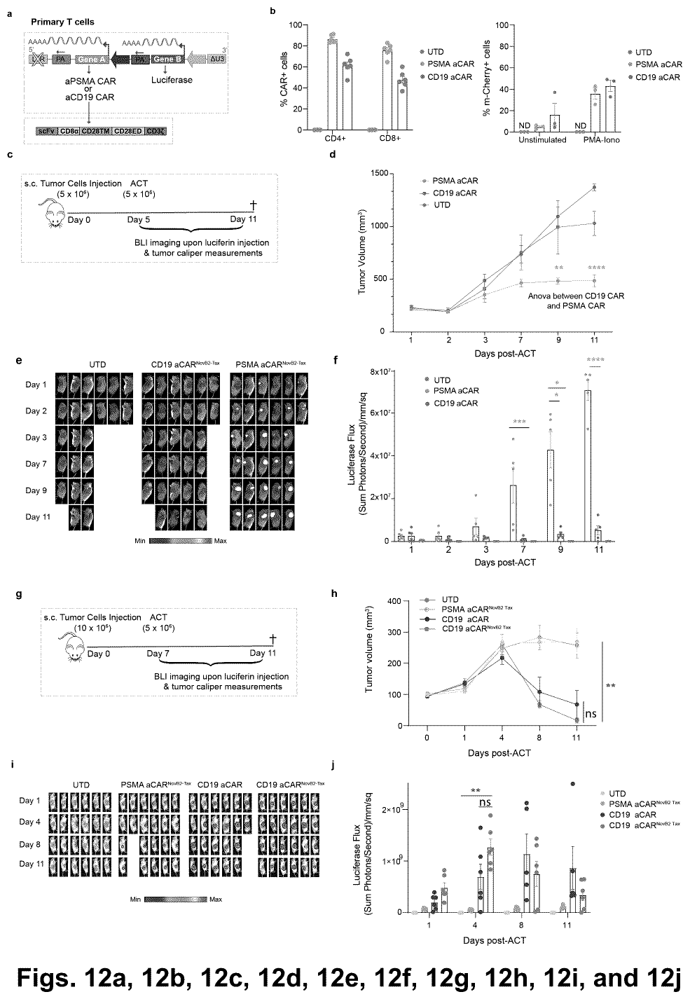

For proof-of-principle in vivo of the antisense lentiviral vector approach, next-generation anti-PSMA and anti-CD19 CAR-T cells with luciferase (for imaging purposes) expressed under 6xNFAT as inducible gene cargo (FIG. 12a) were evaluated. Efficient transduction of primary human T cells was achieved for both of the antisense lentiviral 4G CAR constructs (FIG. 12b, left). Low levels of background mCherry expression were observed in non-activated anti-CD19 CAR-T cells, presumably due to minor tonic signaling, but upon PMA-Iono activation, similar % mCherry expression was observed for both of the CAR constructs (FIG. 12b, right).

For the first in vivo study, NSG mice were inoculated with 5×10⁶ PSMA⁺ PC3-PIP tumor cells and treated on day 5 by peritumoral transfer of 5×10⁶ 4G CAR- or UTD-T cells (FIG. 12c). As expected, the 4G anti-PSMA CAR-T cells, but not the 4G anti-CD19 CAR- nor the UTD-T cells, were able to control tumor growth (FIG. 12d). In addition, luciferase activity upon luciferin injection in mice was only observed for the tumor-infiltrating 4G anti-PSMA CAR-T cells (FIGS. 12e and 12f). Subsequently, the in vivo study was repeated, but next-generation anti-PSMA CAR-T cells generated with antisense versus sense vectors were further compared (FIGS. 5a and 5b). No significant differences were observed in tumor control for antisense versus sense lentiviral vector generated CAR-T cells (FIG. 5c), in line with the in vitro data for this very potent anti-PSMA CAR.

In this example, whether the use of Tax and NovB2 during lentivirus vector production (to increase titers) has any impact on CAR-T cell function was evaluated. It was found that there was no significant difference in tumor control by anti-PSMA CAR-T cells generated with virus produced in the presence or absence of Tax and NovB2 (FIG. 5c). Importantly, however, luciferase activity levels of tumor-infiltrating CAR-T cells, as measured by luminescence imaging upon luciferin injection of the treated mice, were significantly higher for the antisense lentiviral vector generated 4G CAR-T cells (FIGS. 5d and 5e). This observation, which corresponds with the in vitro findings (FIG. 4), is presumably due to a lack of transcriptional interference in the engineered T cells, as occurs upon the use of dual sense lentiviral vectors.

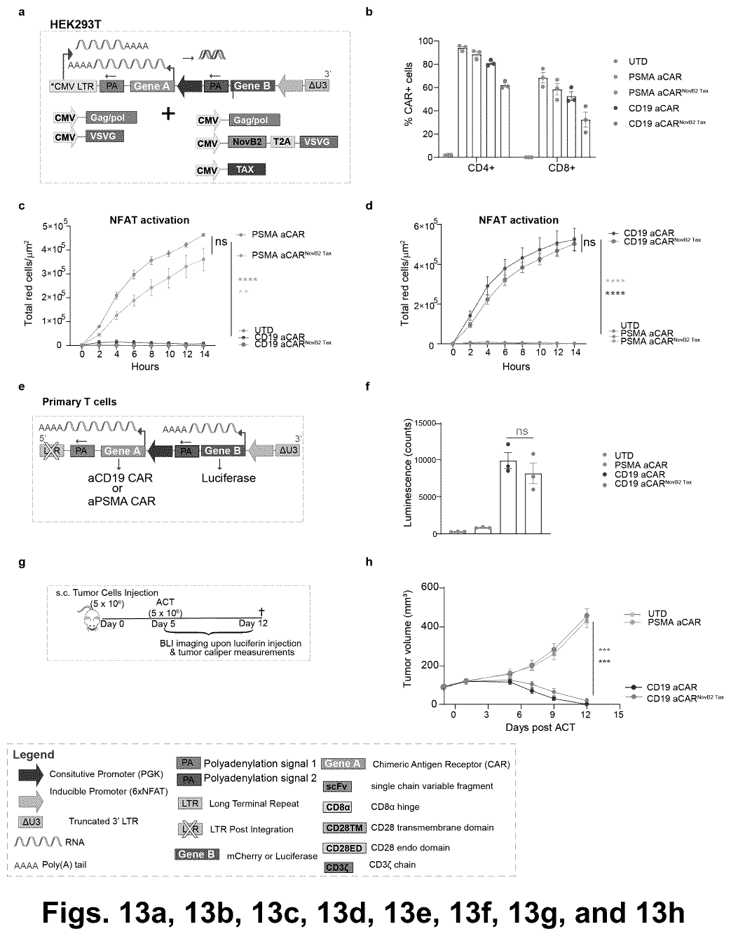

Finally, the next generation CAR-T cells in vivo against a CD19⁺ tumor model was evaluated. Briefly, mice were inoculated with 10×10⁶ Bjab tumor cells and, on day 7, were treated by peritumoral transfer of 5×10⁶ anti-sense lentiviral vector generated 4G CAR-T cells, or UTD-T cells (FIG. 12g). As expected, the anti-CD19 CAR- but not the anti-PSMA-CAR- nor the UTD-T cells were able to control tumor growth. It was observed that there were no significant differences in tumor control (FIG. 12h) nor in NFAT-driven luciferase activity (FIGS. 12iand 12j) for 4G anti-CD19 CAR-T cells generated with virus produced in the presence or absence of Tax and NovB2. It was further shown that the use of NovB2 and Tax during lentivirus vector production (FIG. 13a) had no impact on transduction efficiency (FIG. 13b), the cytolytic capacity of CAR-T cells against target cells (FIG. 13c), the levels of inducibly expressed gene cargo upon CAR-T cell coculture with target cells (FIGS. 13d and 13f), or tumor control by anti-CD19 CAR-T cells (FIGS. 13g and 13h)

### Development of Culture Conditions Suitable for Clinical-Grade Lentivirus Vector Production

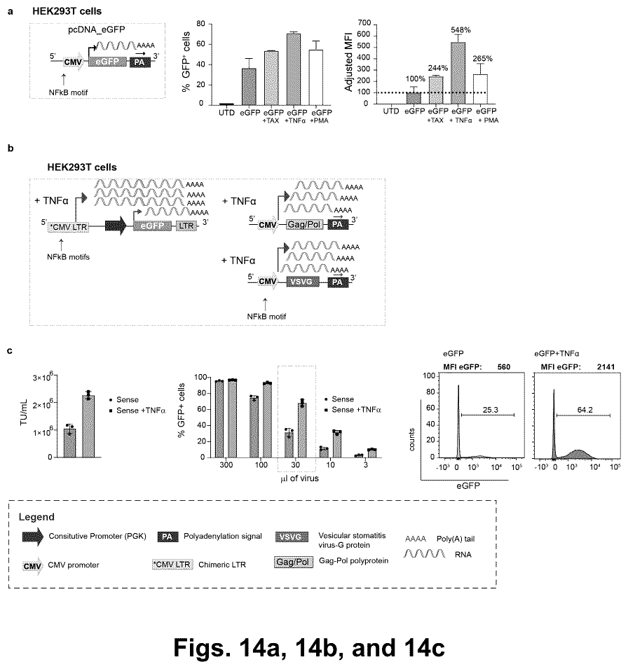

HTLV-Tax has been reported to act on several signaling pathways, among them NF-κB. Although no Tax protein is expected in the lentiviral particle preparation following ultracentrifugation, its tumorigenic potential may raise regulatory concerns for clinical-grade production of lentivirus vectors. It was thus sought to identify a suitable alternative. HEK293T cells were transiently transfected with a suboptimal concentration of pcDNA-eGFP, which harbors a CMV promoter, and the cells were treated with different compounds. At 48 hours post-transfection, an increase was observed in both the percentage and MFI of cells expressing eGFP upon TNFα exposure (FIG. 14a). Encouraged by this observation, the use of TNFα was next tested in the context of sense orientation, single gene-cassette (FIG. 14b) lentivirus vector production in HEK293T cells. A significant increase in viral titer, percentage, and MFI of eGFP⁺ cells was observed (FIG. 14c), presumably due to the effect of TNFα not only on the transfer vector, but also on the envelope and packaging vectors which comprise CMV promoters. Of note, this NFkB-mediated strategy can, in principle, be applied to enhance the production and hence lower the costs of any viral vector comprising NF-κB consensus binding sites in promoter/enhancer regions.

### Evaluation of Clinical-Grade Protocol in the Context of ‘Difficult to Produce’ Lentivirus Vectors

Along with the development of tumor redirected T cells that co-express additional molecules or receptors, gene-downregulation strategies can also be employed to potentiate their function. However, transfer vectors encoding shRNA, which comprise stem-loop structures, are associated with low viral titers due to Dicer processing. Hence, to further validate the use of TNFα and NovB2 to augment viral titers, different transfer vectors comprising a short microRNA (miR)-based short hairpin (sh)RNA hairpin (miR-based shRNA) were developed. Notably, NovB2 has been previously shown to increase the titer of such vectors due to specific inhibition of the canonical activity of Dicer isoforms in processing microRNAs.

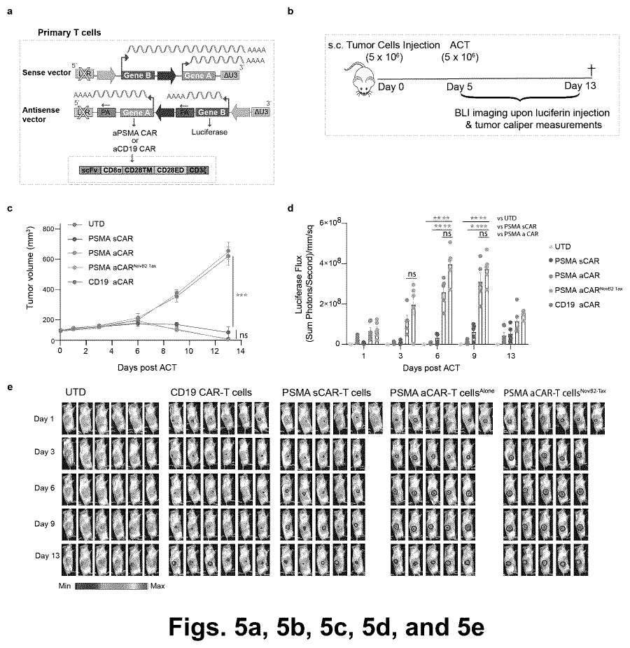

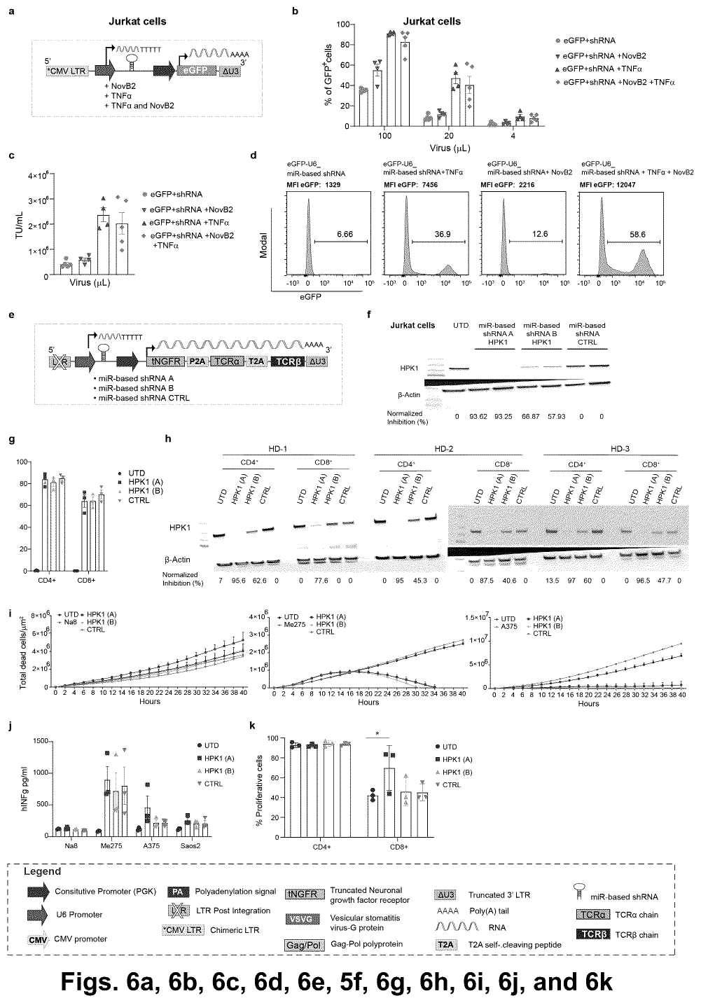

The miR-based shRNA was expressed under the constitutive U6 promoter with eGFP expressed downstream under the PGK promoter (FIG. 6a). Indeed, because the termination of transcription from polymerase III promoters comprises 5 thymidine residues, the vector was built in a dual sense orientation; there is no transcriptional interference to reach a PA site and hence no need to invert the gene-cassette. Upon titration of viral supernatant produced in the presence of NovB2, TNFα, or both, an important gain was observed in transduction efficiency as measured by percentage of eGFP⁺ cells (FIG. 6b), lentiviral titer (FIG. 6c), and relative expression level of eGFP per cell (MFI) (FIG. 6d).

Based on these results, a sense vector was subsequently constructed, having a miR-based shRNA under the U6 promoter targeting a therapeutically relevant target, Hematopoietic Progenitor Kinase 1 (Hpk1), a negative regulator of TCR signaling, also known as Mitogen-Activated Protein Kinase 1 (Map4k1). The miR-based shRNAs were followed by truncated human nerve growth factor receptor (tNGFR), and the HLA-A2/NY-ESO-1₁₅₇₋₁₆₅ restricted TCR, both expressed under the PGK promoter and separated by a T2A element (FIG. 6e). Jurkat cells transduced with this construct showed an efficient knockdown of HPK1 (over 90% reduction by miR-based shRNA ‘A’) (FIG. 6f). Primary T cells were then transduced, and 85% transduction efficiency of primary CD4⁺ T cells, and around 70% for CD8⁺ T cells, as measured by HLA-A2/NY-ESO-1₁₅₇₋₁₆₅ tetramer staining, were observed (FIG. 6g). Efficient transduction was accompanied by strong HPK1 knockdown, similar to the levels observed in Jurkat cells (FIG. 6h). the in vitro function of the TCR-T cells +/−HPK1 knockdown was subsequently evaluated by miR-based shRNA upon coculture with the A2⁺/NY⁺ target cell lines Me275 and A375, as well as the A2⁺/NY⁻ cell line Na8 as a negative control. Others have previously demonstrated that pharmacological inhibition, or full gene knock-out, of HPK1 in CD8⁺ T cells can improve their effector function and ability to control tumors. However, no significant differences were observed in target cell killing (FIG. 6i) nor in IFNg release (FIG. 6j) for the HPK1 knockdown TCR-T cells (HPK1 ‘A’ and ‘B’) versus the control (CTRL) TCR-T cells comprising a scrambled miR-based shRNA, but a higher proliferative capacity for the HPK1 ‘A’ knockdown CD8⁺ T cells was observed (FIG. 6k).

### Evaluation of Clinical-Grade Lentivirus Vector Production Protocol for Antisense Transfer Vectors

The use of TNFα in combination with NovB2 was next tested in the context of the antisense configuration transfer vector encoding mCherry under 6xNFAT and eGFP under PGK (FIG. 7a, left). Similar to when Tax was used, a gain in viral titer was observed in the presence of TNFα alone, but titers were even higher if NovB2 was combined with TNFα (FIG. 7a, middle and right panel).

It is well known that the use of vectors comprising U6-driven shRNAs can be toxic to transfected cells, and polymerase III promoters do not allow for inducible expression of genes of interest. Hence, to overcome this obstacle, an antisense vector was next constructed, comprising a miR-based shRNA under 6xNFAT and eGFP under PGK (FIG. 7b, left) and produced a lentivirus vector using the optimized, clinical-grade production protocol. An important gain was observed in viral titer in the presence of NovB2 alone, or combined with TNFα (FIG. 7b, middle and right panel).

An inverted configuration vector was further evaluated, comprising the anti-PSMA CAR and miR-based shRNA ‘A’ targeting HPK1 under 6xNFAT in primary human T cells (FIG. 7c, left). Upon transduction with lentivirus vector produced in the presence of NovB2 and TNFα, approximately 90% CAR expression by CD4⁺ T cells, and about 60% for CD8⁺ T cells were achieved (FIG. 7c, middle). Moreover, upon 6 hour CAR-T cell triggering with plate-coated anti-F(ab), over 90% HPK1 knockdown was achieved (FIG. 7c, right).

Finally, for the dual anti-sense vector, a miR-based shRNA was cloned, targeting the TCR-alpha chain under an alternative constitutive Polymerase II promoter, SFFV (silencing prone spleen focus forming virus), and eGFP under PGK (FIG. 7d, left). This is a strategy that can be used to abrogate TCR chain mispairing upon engineering of T cells for ACT with an exogenous TCR. Transduced Jurkat cells demonstrated efficient knockdown of the TCR-alpha chain with the dual antisense vector as measured by cell-surface staining with a pan-anti-TCR antibody (FIG. 7d, bottom right).

Thus, this example demonstrated that the use of TNFα during virus production, using antisense (or sense) transfer vectors in which the RSV-based promoter and enhancer at the 5′LTR are replaced with the complete CMV promoter and enhancer (which comprises 4 consensus NF-κB binding motifs), can significantly increase titers. It is likely that the TNFα, in addition to favoring transcription of the transfer vector, also promotes replication of the packaging and envelope vectors. Moreover, the presence of TNFα in the culture media can synergize with NovB2, a protein that can abrogate Dicer mediated dsRNA antiviral response generated during virus production in HEK293T cells. In addition, the protocol, which is feasible for the production of clinical-grade viruses at reduced costs, can be used to generate high titers of ‘difficult to produce’ lentivirus vector such as ones encoding miR-based shRNA. Indeed, NovB2 may further abrogate Dicer mediated processing of such hairpin structures.

### DISCUSSION

In recent years, rational TCR- and CAR-T cell co-engineering strategies have been under extensive investigation to improve responses against solid tumors, either by directly enhancing the intrinsic fitness and function of the T cells themselves or/and by TME reprogramming. In addition to barriers in the TME, the clinical success of T-cell therapy against solid tumors is constrained by adverse patient reactions such as on-target but off-tumor toxicity, as well as cytokine release syndrome by CAR-T cells, and unexpected cross-reactivity by TCR-T cells against vital organs. Hence, important research efforts are also being undertaken in the development of ON- and OFF- and STOP-switches along with gene-modification strategies and optimized vectors to allow tighter control of the biological activities of engineered T cells post-infusion.

The strong safety record of lentiviral vectors coupled with enhanced manufacturing protocols and the high transduction efficiencies make lentivirus vectors an important clinical tool. Given the tremendous potential of lentiviral vectors, further optimization of lentiviral vectors, virus production methods, and transduction strategies are warranted. Here, an antisense transfer vector was developed, allowing efficient constitutive expression of a tumor-directed TCR or CAR and independent co-expression of gene cargo. While the activation inducible promoter 6xNFAT was used to express various gene cargo, including IL-2 and miR-based shRNAs, to knockdown genes of interest, it is also feasible to employ promoters that respond to environmental cues, including hypoxia. Such an approach will be useful, for example, for co-expression of chemokines which can generate a gradient to attract additional lymphocytes into the tumor bed. The development of drug-inducible promoters, like the tetracycline controlled ON system (Tet-ON, of bacterial origin) but comprising non-immunogenic components suitable for the clinic, allowing sufficient expression levels of the target molecule(s) of interest for therapeutic efficacy, will be of great benefit for tighter and safer control of next generation TCR- and CAR-T cells and other cellular therapies.

In this example, side-by-side evaluation with comparative dual forward and bidirectional vectors revealed transcriptional interference for the former and leakiness of the inducible promoter for the latter configuration. However, it was shown that primary human T cells could be efficiently engineered with a lentivirus vector comprising a dual antisense transfer vector encoding a constitutively expressed CAR or TCR and inducible gene cargo without such problems. Moreover, next-generation TCR- and CAR-T cells engineered with the dual antisense lentiviral constructs were validated for functionality both in vitro and in vivo in the context of solid tumor-bearing mice.

While the antisense transfer vector design was limiting to virus production, evidently because of convergent transcription in HEK293T cells, a robust protocol was developed to restore titers. First, it was demonstrated the presence of the RNA interference suppressor protein NovB2, capable of inhibiting isoforms of Dicer, could augment lentiviral titers. The issue that transcriptional interference is limiting to the levels of the ssRNA viral genome available for packaging was subsequently addressed. The Tax protein was first tested, which, amongst a variety of oncogenic properties, can act as a potent transactivator of CMV promoters as they harbor 4 NK-kB binding motifs. Indeed, when the RSV-based promoter and enhancer at the 5′LTR of the transfer vector were replaced with the complete CMV promoter and enhancer, viral titers were increased in the presence of Tax, and to a greater extent when combined with NovB2.

For potential clinical GMP grade production of lentivirus vector, a substitution for Tax was sought. It was demonstrated that the presence of TNFα in the culture supernatant, previously shown to efficiently act on NF-xB binding motifs in a dose-dependent manner (Hellweg, C. E., et al. Ann N Y Acad Sci 1091, 191-204 (2006)), also increased viral titers. Notably, the use of TNFα to increase viral titers may be applicable to other viruses produced from vectors comprising promoters with NF-κB binding motifs. Moreover, TNFα may be useful for increasing plasmid production (i.e., comprising NF-κB binding motifs) in transfected cells.

Recently, an ‘all in one’ dual sense lentiviral vector system was described comprising inducible expression of a gene upstream of a constitutively expressed second gene. However, in line with previous work, the data here indicate transcriptional interference for this design and, consequently, lower gene expression, presumably due to competition for the same PA site and the simultaneous occupancy of the DNA template. This lower expression may be limited to the therapeutic efficacy of the cellular product, such as T cells gene-modified to secrete a decoy molecule targeting an immune checkpoint such as programmed death-protein 1 (PD-1). The enhanced expression of genes from the dual inverted vector is likely due both to a lack of transcriptional interference, as well as the use of the potent BGH polyadenylation signal (West, S. & Proudfoot, N.J. Mol Cell 33, 354-364 (2009)).

A bi-directional transfer vector design was further tested, but expression of the inducible gene in non-activated cells was observed. While it may be possible to abrogate leakiness by further buffering the two promoters, this will be limited to the size of the genes that can subsequently be accommodated; beyond a genomic load of 10,000 bp lentiviral vectors become increasingly inefficient.

Taken together, this example presents an improved dual antisense transfer vector and accompanying lentivirus vector production protocol enabling efficient transduction of primary human T cells with a constitutively expressed tumor-targeting receptor along with independent, activation-inducible co-expression of gene cargo. The functionality of the dual inverted vector encoding either a CAR or a TCR under PGK and various gene cargo under 6xNFAT, including IL-2 and miR-based shRNA targeting HPK1, was demonstrated. It was further demonstrated proof-of-principle for the use of the dual inverted vector for generating 4G CAR-T cells for use in vivo. It was shown that the inducible gene cargo (luciferase) was expressed by T cells in tumors only if a target antigen for the CARs was present. Notably, the overall approach is universal in that it can be applied to the engineering of other cell types, alternative polymerase II promoters, and different engineering purposes in the context of other diseases. Importantly, the strategy can lower costs due to the use of a single vector and higher titers achieved, and it holds important promise towards effective and safety-enhanced next generation cellular therapies reaching the clinic.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A polynucleotide, comprising:
a first gene cassette comprising at least a first polynucleotide sequence operably linked to a constitutive promoter or an inducible promoter; and
a second gene cassette comprising at least a second polynucleotide sequence operably linked to a second constitutive promoter or a second inducible promoter,
wherein both the first gene cassette and the second gene cassette are in antisense orientation and in the same strand of the polynucleotide.

2. The polynucleotide of claim 1, wherein the first polynucleotide sequence is operably linked to the constitutive promoter.

3. The polynucleotide of claim 1, wherein the second polynucleotide sequence is operably linked to the second inducible promoter.

4. The polynucleotide of claim 1, wherein the first gene cassette or the second gene cassette comprises two or more polynucleotide sequences; optionally wherein the two or more polynucleotide sequences are separated by a T2A or P2A element.

5. The polynucleotide of claim 1, wherein the first gene cassette further comprises a third polynucleotide sequence that is separated from the first polynucleotide sequence by a T2A or P2A element; or wherein the second gene cassette further comprises a fourth polynucleotide sequence that is separated from the second polynucleotide sequence by a T2A or P2A element.

6. The polynucleotide of claim 1, wherein the first gene cassette comprises one or more genes of a CAR, a co-stimulatory CAR, a TCR, a cellular elimination tag, and a decoy that are regulated by the constitutive promoter; and/or wherein the second gene cassette comprises one or more genes of a cytokine, a Flt3L, a LIGHT, a chemokine, a co-stimulatory ligand, a decoy, a dominant negative receptor, a signal switch receptor, and a gene knockdown that are regulated by the second inducible promoter.

7. The polynucleotide of claim 1, wherein the first gene cassette comprises one or more genes of a cytokine, a chemokine, a co-stimulatory ligand, a decoy, a Trap, a dominant negative receptor, a signal switch receptor, and a gene knockdown that are regulated by the inducible promoter; and/or wherein the second gene cassette comprises one or more genes of a cytokine, a chemokine, a co-stimulatory ligand, a decoy, a dominant negative receptor, a signal switch receptor, and a second gene knockdown to complement a gene in the first gene cassette, wherein the one or more genes are regulated by the second inducible promoter.

8. The polynucleotide of claim 1, wherein the first polynucleotide sequence encodes (i) a CAR or TCR or (ii) a gene cargo.

9. (canceled)

10. The polynucleotide of claim 1, wherein the first gene cassette is located in 5′ of the second gene cassette.

11. The polynucleotide of claim 1, further comprising a polyadenylation (PA) signal located between the first gene cassette and the second gene cassette, whereby independent RNAs are transcribed and separately translated.

12. The polynucleotide of claim 1, wherein the first gene cassette and the second gene cassette are arranged between a 5′LTR and a 3′ LTR.

13. The polynucleotide of claim 11, wherein the 3′ LTR is a self-inactivating (SIN) LTR.

14. The polynucleotide of claim 1, wherein the constitutive promoter comprises any one of a human phosphoglycerate kinase-1 (PGK) promoter, a cytomegalovirus (CMV) immediate-early gene promoter, an elongation factor 1 alpha (EF1α) promoter, a ubiquitin-C(UBQ-C) promoter, a cytomegalovirus (CAG) enhancer/chicken beta-actin promoter, a polyoma enhancer/herpes simplex thymidine kinase (MC1) promoter, a beta-actin (β-ACT) promoter, a simian virus 40 (SV40) promoter, a dl587rev primer-binding site substituted (MND) promoter, and a combination thereof.

15. The polynucleotide of claim 1, wherein the inducible promoter comprises an NFAT promoter.

16. The polynucleotide of claim 6, wherein the CAR or TCR binds to an antigen selected from: prostate-specific membrane antigen (PSMA), Carcinoembryonic Antigen (CEA), CD19, CD20, CD22, ROR1, mesothelin, CD333/IL3Ra, c-Met, Glycolipid F77, EGFRvIII, GD-2, NY-ESO-1 TCR, ERBB2, BIRC5, CEACAM5, WDR46, BAGE, CSAG2, DCT, MAGED4, GAGE1, GAGE2, GAGE3, GAGE4, GAGE5, GAGE6, GAGE7, GAGE 8, IL13RA2, MAGEA1, MAGEA2, MAGE A3, MAGEA4, MAGEA6, MAGEA9, MAGE A 10, MAGEA12, MAGEB1, MAGEB2, MAGEC2, TP53, TYR, TYRP1, SAGEl, SYCP1, SSX2, SSX4, KRAS, PRAME, NRAS, ACTN4, CTNNB1, CASP8, CDC27, CDK4, EEF2, FN1, HSPA1B, LPGAT1, MEl, HHAT, TRAPPC1, MUM3, MYOIB, PAPOLG, OS9, PTPRK, TPI1, ADFP, AFP, AIM2, ANXA2, ART4, CLCA2, CPSF1, PPIB, EPHA2, EPHA3, FGF5, CA9, TERT, MGAT5, CEL, F4.2, CAN, ETV6, BIRC7, CSF1, OGT, MUC1, MUC2, MUM1, CTAG1A, CTAG2, CTAG, MRPL28, FOLH1, RAGE, SFMBT1, KAAG1, SART1, TSPYL1, SART3, SOX10, TRG, WTI, TACSTD1, SILV, SCGB2A2, MC1R, MLANA, GPR143, OCA2, KLK3, SUPT7L, ARTC1, BRAF, CASP5, CDKN2A, UBXD5, EFTUD2, GPNMB, NFYC, PRDX5, ZUBR1, SIRT2, SNRPD1, HERV-K-MEL, CXorffi1, CCDC1 10, VENTXP1, SPA17, KLK4, ANKRD30A, RAB38, CCND1, CYP1B1, MDM2, MMP2, ZNF395, RNF43, SCRN1, STEAPI, 707-AP, TGFBR2, PXDNL, AKAP13, PRTN3, PSCA, RHAMM, ACPP, ACRBP, LCK, RCVRN, RPS2, RPLIOA, SLC45A3, BCL2L1, DKK1, ENAH, CSPG4, RGS5, BCR, BCR-ABL, ABL-BCR, DEK, DEK-CAN, ETV6-AML1, LDLR-FUT, NPM1-ALK1, PML-RARA, SYT-SSX1, SYT-SSX2, FLT3, ABL1, AML1, LDLR, FUT1, NPM1, ALK, PML1, RARA, SYT, SSX1, MSLN, UBE2V1, HNRPL, WHSC2, EIF4EBP1, WNK2, OAS3, BCL-2, MCL1, CTSH, ABCC3, BST2, MFGE8, TPBG, FMOD, XAGE1, RPSA, COTL1, CALR3, PA2G4, EZH2, FMNL1, HPSE, APC, UBE2A, BCAP31, TOP2A, TOP2B, ITGB8, RPA1, ABI2, CCNI, CDC2, SEPT2, STAT1, LRP1, ADAM17, JUP, DDR1, ITPR2, HMOX1, TPM4, BAAT, DNAJC8, TAPBP, LGALS3BP, PAGE4, PAK2, CDKN1A, PTHLH, SOX2, SOX11, TRPM8, TYMS, ATIC, PGK1, SOX4, TOR3A, TRGC2, BTBD2, SLBP, EGFR, IER3, TTK, LY6K, IGF2BP3, GPC3, SLC35A4, HSMD, H3F3A, ALDH1A1, MFI2, MMP14, SDCBP, PARP12, MET, CCNB1, PAX3-FKHR, PAX3, FOXO1, XBP1, SY D1, ETV5, HSPA1A, HMHA1, TRIM68, and a fragment thereof.

17. The polynucleotide of claim 8, wherein the gene cargo is selected from IL-2, IL2v, IL-12, IL-15, IL-18, IL21, IFNγ, IL7, IL23, IL33, GM-CSF, Flt3-L, 41BB-L, CD40-L, TGFb, VEGF, IL10, PD1, TGFβR, a dominant negative receptor, a signal switch receptor, CCL5, CXCL9, CXCL10, XCL1, and a combination thereof.

18. The polynucleotide of claim 1, wherein; (i) the gene cargo comprises an shRNA, miRNA, or a sequence enabling down-regulation of a target gene; and/or (ii) the target gene comprises HPK1 or Cb1b.

19. (canceled)

20. A vector comprising the polynucleotide of claim 1.

21. The vector of claim 18, wherein the vector is a retroviral vector or a lentiviral vector.

22. The vector of claim 19, wherein the lentiviral vector is selected from human immunodeficiency virus 1 (HIV-1), human immunodeficiency virus 2 (HIV-2), visna-maedi virus (VMV), caprine arthritis-encephalitis virus (CAEV), equine infectious anemia virus (EIAV), and feline immunodeficiency virus (FIV).

23. A viral particle or virus-like particle (VLP) comprising the polynucleotide of claim 1.

24. A cell comprising the polynucleotide of claim 1.

25. (canceled)

26. The cell of claim 24 or 25, wherein the cell is autologous or allogeneic.

27. A pharmaceutical composition comprising the polynucleotide of claim 1.

28. A kit comprising the polynucleotide of claim 1.

29. A method for preparing an immune effector cell expressing a CAR or TCR, comprising introducing into an immune effector cell the polynucleotide of claim 1.

30. (canceled)

31. (canceled)

32. (canceled)

33. A method of treating cancer in a subject, comprising administering to the subject a therapeutically effective amount of the polynucleotide of claim 1.

34. (canceled)

35. (canceled)

36. (canceled)

37. (canceled)
